SEQUENCE LISTING (40 sequences)

SEQ ID NO: 1 - DNA, 1453 bp, synthetic construct
  source 1..1453: mol_type = other DNA
cacggctgaagagatgagaccatcaggccagctccccctaacggggctgctattcttttctcttattcccagtcaactatgccagatctgtgtggtaaatgaaacgaacttctggcgtctagaacctctgataaacacgatgctgaatgcaaactacaccaaagggaccctaggtctcaatgtcctgttgaccttgagacttgttgggattaagaatcaaagcctagaacaacagctgagtcaacaaatcaaagaagacataaacagcggagagatggagttaacctcgggagagcttgccttgggtattctgggtttgggagcatgtgaaaatcaggatgaagagtttatacgtggtgctcgtctggttagcaaacttgaagccaaattccaagcagaaattcagaacatggaggcacatgacggcaacccactgaccaactactaccagctcagcctggccctgctggccttgtgtctgttcaacggcagctattcagtcaccagtgttacctgttacttcactcctgaaaataagaactattattttggggaccagttctcagtagatactggagcgatggctgttcttgctttaacatgtgtgcagaagaatacacaaaagaatatggacatgaacgcagagggtaaaatcagtaattacgtggcgtcactggcaaacaagatccaggctgagagaaaacagggtctctttggaaacatatttagcacgggagaagctatgcaggctctctttgtctcatcggactactacaaaaatgaagcaaattgcagagaaactctacgtgcagtatttgacaacatttctcggggagcattttacttgccgattactgcagcccagatcttacctgccttgatgggaaagacctatctggatgtcaccaacccttcttgtggcctcaatccagtcaaattcaacacctccactgaaaagcctggtactgtgacacctacaaccgcacccttgaatatcttagtcaaatactctgtgcgaatcaacaagacaagtcacactgaagtcactgtgagaaagggttctgtcttcctcgacgtgatgaaggctgctcaggagaaaaatgagaccctatttcgcttcacagtggaggagacctcgtggggcccctacatcacctctgttcagggcataaaggccagcaataatgacagaacctactggaagcttctgagcaacggccagccactgagccaaggagctgggagtcatgttgtccaaaatggagacaatttggaggttcgttggagcaaatactaataaattcaaaccttcctcagctgggtctgagccttttgtggtacagttctatgtggaaattgatcttacatgttccttttcatttatatctcaatcctcacagcacatcaataacctctacttctctctttgagtgtccaataaaagaccaacagc

SEQ ID NO: 2 - DNA, 2236 bp, synthetic construct
  source 1..2236: mol_type = other DNA
ctgtcccaggccggggtgggcagtttcaggggctccttctgggaggggtagcaggtaggcaccctagcctaccgtgtctaccgagggcatacagacaggcgtggctggacagactcagcagtcagacgttctgcacccccatccagggctcttcgggcggcggggaaagtgtggctggccgcactcgcccctgcccccgcctccaggaaggcccgcgctgctctgcccttccttcccctgttctttggcctccctttcccctcagctacagaccttttcccttttccctctgctgacgtagcaatgtgctgccgggcgtggagggattaatcagtgacaggaagctgcctctttcggagcagtgaccactgtggtcaggagagcctctctctacccggccagccctaggagcctgtcccgctccccgcctgctgtccaccccctgctcctgccatggggcacctcggggccctcctcttcctgctggggggcctgggagcgctagccaacatctgcgagataaccgaggtggacagcacgctggtggagaggctgggccagcgcctcttgccctggatggaccggctctccccggagcagctgaaccccagtatctacgtgggcctgcgcctctcgagcctgcaggctggggccaaggaggcccactacctgcacagcctcaagctcagctaccagcagagcctcctgaggcctgcctccaacaaggatgacaatgactccgaggccaagccctctatgggccagctggccctctacctgctggctctccgggccaactgcgagttcatcggaggccgcaagggggacaggctggtctcccagctgaagcggttcctggaggacgagaagagggccatcgggcacaaccaccagggtcacccccgcaccagctactaccagtacagcctgggcatcctggccctgtgtgtccaccagaagcgagtccacgacagtgtggtgggcaagctcctgtacgccgtggaacacaagccgcatctcctgcaggaccacgtctctgtggacaccatggccatggcaggcatggccttctcctgtctggagctgtccaacctcaaccccaagcagagaaaccggatcaacctggccctcaagagagtgcaagagaagatcctgaaggcccagaccccagagggctacttcgggaatgtctacagcacccctctggctttgcagttgctgatgggctccctcaggccctcggtggagctgggcacagcctgccttaaagccaaggctgctctgcaggccagcctacagcacaagaccttccagaaccctctcatgatctctcagctgctgcctgtcctgaaccagaagagctatgtggatctcatctccccagactgccaggctccaagagccctgttggaaccggctctggagaccccgccacaggccaaagtcccgaagttcattgacgtcttgctgaaggtctccggcatctccccttcatacagacactctgtctctgtccctgctggctcctccctggaagacatcctgaagaacgcccaggagcatggaagattcaggtttagaacacaggcctccctgtctggccccttcctgacctccgtgctggggagaaaggctggggaacgtgagttctggcaggtcctccgagatcctgacacccccttgcagcaaggtattgctgactacagacccaaggatggagagaccatcgagctgaggctggttggctggtagccccccaggctcacccacgccgctgcctcccacacttcctgtctcccaccctccctccagacaccccggtaatgggcgcacgcctgatcctgctgccttctcttacacacttgtccctggatcccacagccgccagccctgcgggagtcctaaaccaccgcccacctggagcagggagcccagcacctccccgaggaggtctgtctgcctgggtctggtcccacctggcccggcgggtgtcccgagaaggtcactcacaggccggagggcgtaaacgtctcggactcctccccggccgcctcgtgagtaccgctcgctctgcgatgggggtcgcttgaggacagccctgcggccctgaccctagctctccactctgtcgtccaggctgccgccccagagccggctgcgttgtagcggctggtgagggtttctgcagagctctcagctcacctctagaaggattaaagagttggttgtgcacgtg

SEQ ID NO: 3 - protein, 427 aa, synthetic construct
MRPSGQLPLTGLLFFSLIPSQLCQICVVNETNFWRLEPLINTMLNANYTKGTLGLNVLLTLRLVGIKNQSLEQQLSQQIKEDINSGEMELTSGELALGILGLGACENQDEEFIRGARLVSKLEAKFQAEIQNMEAHDGNPLTNYYQLSLALLALCLFNGSYSVTSVTCYFTPENKNYYFGDQFSVDTGAMAVLALTCVQKNTQKNMDMNAEGKISNYVASLANKIQAERKQGLFGNIFSTGEAMQALFVSSDYYKNEANCRETLRAVFDNISRGAFYLPITAAQILPALMGKTYLDVTNPSCGLNPVKFNTSTEKPGTVTPTTAPLNILVKYSVRINKTSHTEVTVRKGSVFLDVMKAAQEKNETLFRFTVEETSWGPYITSVQGIKASNNDRTYWKLLSNGQPLSQGAGSHVVQNGDNLEVRWSKY

SEQ ID NO: 4 - protein, 432 aa, synthetic construct
MGHLGALLFLLGGLGALANICEITEVDSTLVERLGQRLLPWMDRLSQEQLNPSIYVGLRLSSLQAGAKEAHYLHSLKLSYQQSLLRPASNKDDNDSEAKPSMGQLALYLLALRANCEFIGGRKGDRLVSQLKRFLEDEKRAIGHNHQGHPRTSYYQYSLGILALCVHQKRVHDSVVGKLLYAVEHKPHLLQDHVSVDTMAMAGMAFSCLELSNLNPKQRNRINLALKRVQEKILKAQTPEGYFGNVYSTPLALQLLMGSLRPSVELGTACLKAKAALQASLQHKTFQNPLMISQLLPVLNQKSYVDLISPDCQAPRALLEPALETPPQAKVPKFIDVLLKVSGISPSYRHSVSVPAGSSLEDILKNAQEHGRFRFRTQASLSGPFLTSVLGRKAGEREFWQVLRDPDTPLQQGIADYRPKDGETIELRLVGW

SEQ ID NO: 5 - protein, 404 aa, synthetic construct
QICVVNETNFWRLEPLINTMLNANYTKGTLGLNVLLTLRLVGIKNQSLEQQLSQQIKEDINSGEMELTSGELALGILGLGACENQDEEFIRGARLVSKLEAKFQAEIQNMEAHDGNPLTNYYQLSLALLALCLFNGSYSVTSVTCYFTPENKNYYFGDQFSVDTGAMAVLALTCVQKNTQKNMDMNAEGKISNYVASLANKIQAERKQGLFGNIFSTGEAMQALFVSSDYYKNEANCRETLRAVFDNISRGAFYLPITAAQILPALMGKTYLDVTNPSCGLNPVKFNTSTEKPGTVTPTTAPLNILVKYSVRINKTSHTEVTVRKGSVFLDVMKAAQEKNETLFRFTVEETSWGPYITSVQGIKASNNDRTYWKLLSNGQPLSQGAGSHVVQNGDNLEVRWSKY

SEQ ID NO: 6 - protein, 414 aa, synthetic construct
NICEITEVDSTLVERLGQRLLPWMDRLSQEQLNPSIYVGLRLSSLQAGAKEAHYLHSLKLSYQQSLLRPASNKDDNDSEAKPSMGQLALYLLALRANCEFIGGRKGDRLVSQLKRFLEDEKRAIGHNHQGHPRTSYYQYSLGILALCVHQKRVHDSVVGKLLYAVEHKPHLLQDHVSVDTMAMAGMAFSCLELSNLNPKQRNRINLALKRVQEKILKAQTPEGYFGNVYSTPLALQLLMGSLRPSVELGTACLKAKAALQASLQHKTFQNPLMISQLLPVLNQKSYVDLISPDCQAPRALLEPALETPPQAKVPKFIDVLLKVSGISPSYRHSVSVPAGSSLEDILKNAQEHGRFRFRTQASLSGPFLTSVLGRKAGEREFWQVLRDPDTPLQQGIADYRPKDGETIELRLVGW

SEQ ID NO: 7 - protein, 24 aa, synthetic construct
MKFFAIAALFAAAAVAQPNVISKR

SEQ ID NO: 8 - protein, 16 aa, synthetic construct
MKFFAIAALFAAAAVA

SEQ ID NO: 9 - DNA, 1215 bp, synthetic construct
  source 1..1215: mol_type = other DNA
cagatctgtgtggtaaatgaaacgaacttctggcgtctagaacctctgataaacacgatgctgaatgcaaactacaccaaagggaccctaggtctcaatgtcctgttgaccttgagacttgttgggattaagaatcaaagcctagaacaacagctgagtcaacaaatcaaagaagacataaacagcggagagatggagttaacctcgggagagcttgccttgggtattctgggtttgggagcatgtgaaaatcaggatgaagagtttatacgtggtgctcgtctggttagcaaacttgaagccaaattccaagcagaaattcagaacatggaggcacatgacggcaacccactgaccaactactaccagctcagcctggccctgctggccttgtgtctgttcaacggcagctattcagtcaccagtgttacctgttacttcactcctgaaaataagaactattattttggggaccagttctcagtagatactggagcgatggctgttcttgctttaacatgtgtgcagaagaatacacaaaagaatatggacatgaacgcagagggtaaaatcagtaattacgtggcgtcactggcaaacaagatccaggctgagagaaaacagggtctctttggaaacatatttagcacgggagaagctatgcaggctctctttgtctcatcggactactacaaaaatgaagcaaattgcagagaaactctacgtgcagtatttgacaacatttctcggggagcattttacttgccgattactgcagcccagatcttacctgccttgatgggaaagacctatctggatgtcaccaacccttcttgtggcctcaatccagtcaaattcaacacctccactgaaaagcctggtactgtgacacctacaaccgcacccttgaatatcttagtcaaatactctgtgcgaatcaacaagacaagtcacactgaagtcactgtgagaaagggttctgtcttcctcgacgtgatgaaggctgctcaggagaaaaatgagaccctatttcgcttcacagtggaggagacctcgtggggcccctacatcacctctgttcagggcataaaggccagcaataatgacagaacctactggaagcttctgagcaacggccagccactgagccaaggagctgggagtcatgttgtccaaaatggagacaatttggaggttcgttggagcaaatactaa

SEQ ID NO: 10 - DNA, 1245 bp, synthetic construct
  source 1..1245: mol_type = other DNA
aacatctgcgagataaccgaggtggacagcacgctggtggagaggctgggccagcgcctcttgccctggatggaccggctctcccaggagcagctgaaccccagtatctacgtgggcctgcgcctctcgagcctgcaggctggggccaaggaggcccactacctgcacagcctcaagctcagctaccagcagagcctcctgaggcctgcctccaacaaggatgacaatgactccgaggccaagccctctatgggccagctggccctctacctgctggctctccgggccaactgcgagttcatcggaggccgcaagggggacaggctggtctcccagctgaagcggttcctggaggacgagaagagggccatcgggcacaaccaccagggtcacccccgcaccagctactaccagtacagcctgggcatcctggccctgtgtgtccaccagaagcgagtccacgacagtgtggtgggcaagctcctgtacgccgtggaacacaagccgcatctcctgcaggaccacgtctctgtggacaccatggccatggcaggcatggccttctcctgtctggagctgtccaacctcaaccccaagcagagaaaccggatcaacctggccctcaagagagtgcaagagaagatcctgaaggcccagaccccagagggctacttcgggaatgtctacagcacccctctggctttgcagttgctgatgggctccctcaggccctcggtggagctgggcacagcctgccttaaagccaaggctgctctgcaggccagcctacagcacaagaccttccagaaccctctcatgatctctcagctgctgcctgtcctgaaccagaagagctatgtggatctcatctccccagactgccaggctccaagagccctgttggaaccggctctggagaccccgccacaggccaaagtcccgaagttcattgacgtcttgctgaaggtctccggcatctccccttcatacagacactctgtctctgtccctgctggctcctccctggaagacatcctgaagaacgcccaggagcatggaagattcaggtttagaacacaggcctccctgtctggccccttcctgacctccgtgctggggagaaaggctggggaacgtgagttctggcaggtcctccgagatcctgacacccccttgcagcaaggtattgctgactacagacccaaggatggagagaccatcgagctgaggctggttggctggtag

SEQ ID NO: 11 - DNA, 1287 bp, synthetic construct
  source 1..1287: mol_type = other DNA
atgaagtttttcgccattgccgccctatttgccgccgctgccgttgcccagcctaacgtcatctcgaagcggcagatctgtgtggtaaatgaaacgaacttctggcgtctagaacctctgataaacacgatgctgaatgcaaactacaccaaagggaccctaggtctcaatgtcctgttgaccttgagacttgttgggattaagaatcaaagcctagaacaacagctgagtcaacaaatcaaagaagacataaacagcggagagatggagttaacctcgggagagcttgccttgggtattctgggtttgggagcatgtgaaaatcaggatgaagagtttatacgtggtgctcgtctggttagcaaacttgaagccaaattccaagcagaaattcagaacatggaggcacatgacggcaacccactgaccaactactaccagctcagcctggccctgctggccttgtgtctgttcaacggcagctattcagtcaccagtgttacctgttacttcactcctgaaaataagaactattattttggggaccagttctcagtagatactggagcgatggctgttcttgctttaacatgtgtgcagaagaatacacaaaagaatatggacatgaacgcagagggtaaaatcagtaattacgtggcgtcactggcaaacaagatccaggctgagagaaaacagggtctctttggaaacatatttagcacgggagaagctatgcaggctctctttgtctcatcggactactacaaaaatgaagcaaattgcagagaaactctacgtgcagtatttgacaacatttctcggggagcattttacttgccgattactgcagcccagatcttacctgccttgatgggaaagacctatctggatgtcaccaacccttcttgtggcctcaatccagtcaaattcaacacctccactgaaaagcctggtactgtgacacctacaaccgcacccttgaatatcttagtcaaatactctgtgcgaatcaacaagacaagtcacactgaagtcactgtgagaaagggttctgtcttcctcgacgtgatgaaggctgctcaggagaaaaatgagaccctatttcgcttcacagtggaggagacctcgtggggcccctacatcacctctgttcagggcataaaggccagcaataatgacagaacctactggaagcttctgagcaacggccagccactgagccaaggagctgggagtcatgttgtccaaaatggagacaatttggaggttcgttggagcaaatactaa

SEQ ID NO: 12 - DNA, 1287 bp, synthetic construct
  source 1..1287: mol_type = other DNA
atgaagttcttcgccattgccgccctatttgccgccgccgccgtcgcccagcccaacgtcatctccaagcgccagatctgcgtcgtcaacgagaccaacttctggcgcctcgagcccctcatcaacaccatgctcaacgccaactacaccaagggtaccctcggtctcaacgtcctcctcaccctccgcctcgtcggtatcaagaaccagtccctcgagcagcagctctcccagcagatcaaggaggacatcaactccggtgagatggagctcacctccggtgagctcgccctcggtatcctcggtctcggtgcctgcgagaaccaggacgaggagttcatccgcggtgcccgcctcgtctccaagctcgaggccaagttccaagccgagatccaaaacatggaggcccacgacggtaaccccctcaccaactactaccagctctccctcgccctcctcgccctctgcctcttcaacggttcctactccgtcacctccgtcacctgctacttcacccccgagaacaagaactactacttcggtgaccagttctccgtcgacaccggtgccatggccgtcctcgccctcacctgcgtccaaaagaacacccagaagaacatggacatgaacgccgagggtaaaatctccaactacgtcgcctccctcgccaacaagatccaagccgagcgcaagcagggtctcttcggtaacatcttctccaccggtgaggccatgcaagccctcttcgtctcctccgactactacaagaacgaggccaactgccgcgagaccctccgcgccgtcttcgacaacatctcccgcggtgccttctacctccccatcaccgccgcccagatcctccccgccctcatgggtaaaacctacctcgacgtcaccaacccctcctgcggtctcaaccccgtcaagttcaacacctccaccgagaagcccggtaccgtcacccccaccaccgcccccctcaacatcctcgtcaagtactccgtccgcatcaacaagacctcccacaccgaggtcaccgtccgcaagggttccgtcttcctcgacgtcatgaaggccgcccaggagaagaacgagaccctcttccgcttcaccgtcgaggagacctcctggggtccctacatcacctccgtccaaggtatcaaggcctccaacaacgaccgcacctactggaagctcctctccaacggtcagcccctctcccagggtgccggttcccacgtcgtccaaaacggtgacaacctcgaggtccgctggtccaagtactaa

SEQ ID NO: 13 - DNA, 1287 bp, synthetic construct
  source 1..1287: mol_type = other DNA
atgaagttttttgccattgctgccttatttgccgcggctgccgtcgcccagcctaacgtcatctccaagagacagatctgtgtcgtcaacgagaccaacttctggcgtctggagccgttgatcaacaccatgctcaacgccaactacaccaagggtaccctcggtctcaacgtcctcctcaccctccgcctcgtcggtatcaagaaccagtccctcgagcagcagctctcccagcagatcaaggaggacatcaactccggtgagatggagctcacctccggtgagctcgccctcggtatcctcggtctcggtgcctgcgagaaccaggacgaggagttcatccgcggtgcccgcctcgtctccaagctcgaggccaagttccaggccgagatccagaacatggaggcccacgacggtaaccccctcaccaactactaccagctctccctcgccctcctcgccctctgcctcttcaacggttcctactccgtcacctccgtcacctgctacttcacccccgagaacaagaactactacttcggtgaccagttctccgtcgacaccggtgccatggccgtcctcgccctcacctgcgtccagaagaacacccagaagaacatggacatgaacgccgagggtaagatctccaactacgtcgcctccctcgccaacaagatccaggccgagcgcaagcagggtctcttcggtaacatcttctccaccggtgaggccatgcaggccctcttcgtctcctccgactactacaagaacgaggccaactgccgcgagaccctccgcgccgtcttcgacaacatctcccgcggtgccttctacctccccatcaccgccgcccagatcctccccgccctcatgggtaagacctacctcgacgtcaccaacccctcctgcggtctcaaccccgtcaagttcaacacctccaccgagaagcccggtaccgtcacccccaccaccgcccccctcaacatcctcgtcaagtactccgtccgcatcaacaagacctcccacaccgaggtcaccgtccgcaagggttccgtcttcctcgacgtcatgaaggccgcccaggagaagaacgagaccctcttccgcttcaccgtcgaggagacctcctggggtccctacatcacctccgtccagggtatcaaggcctccaacaacgaccgcacctactggaagctcctctccaacggtcagcccctctcccagggtgccggttcccacgtcgtccagaacggtgacaacctcgaggtccgctggtccaagtactaa

SEQ ID NO: 14 - DNA, 1287 bp, synthetic construct
  source 1..1287: mol_type = other DNA
atgaagttttttgccattgctgccttatttgccgcggctgccgtcgcccagcctaacgtcatctccaagagacagatctgtgtcgtcaacgagaccaacttctggcgtctggagccgttgattaacaccatgttgaacgccaactacaccaaaggcacgctcggtctcaatgttctgctcaccctccgtctggtcggtatcaagaaccaaagcctagaacaacagctctcccagcaaatcaaagaggacatcaacagcggcgagatggaactaacttccggcgagcttgcgctcggtatcctcggtctcggtgcttgcgagaatcaagatgaagaattcattcgtggcgctagattggtttcgaagttggaggccaagttccaggcggagatccagaacatggaggcacatgacggtaaccctctcaccaactactaccagctctccctcgcactacttgcattgtgcctcttcaacggcagctatagcgttacttccgtcacctgttatttcacccccgagaacaagaactactatttcggtgaccagttctctgtcgacactggtgccatggccgttctcgccctcacctgcgtacagaagaacacacagaagaacatggacatgaacgccgagggcaagatctcgaactacgttgcttccctcgccaacaagatccaagccgagaggaagcaaggcctattcggcaacatcttctccaccggcgaggccatgcaggcgctgtttgtctcttcggactactataagaacgaggcaaattgccgtgagactttgcgcgctgtgttcgacaacatctcccgcggtgccttctacctgcctatcaccgccgcccagattctgcccgctctcatgggaaagacctacctcgacgtcaccaacccctcctgcggtctcaaccccgtcaagttcaataccagcaccgagaagcctggtaccgtcacgccaactactgctcctctcaacatccttgtcaagtactcggtccgcattaacaagaccagccacaccgaggtcaccgtccgcaagggctccgtcttcttggatgttatgaaggccgcccaggagaaaaacgagaccctcttccgcttcaccgtggaggagacctcctggggtccctacatcacatccgttcagggtatcaaagcttccaataatgaccgcacctactggaagctcctctccaacggccagccactgtcgcaaggtgctggttctcatgtggttcagaacggcgacaacctggaggtccgctggagcaagtactaa

SEQ ID NO: 15 - DNA, 1287 bp, synthetic construct
  source 1..1287: mol_type = other DNA
atgaagttctttgccattgctgccttatttgccgcggctgccgtcgcccagcctaacgtcatctccaagagacagatctgtgtcgtcaacgagaccaacttctggcgtctggagccgttgattaacaccatgttgaacgccaactacaccaaaggcacgctcggtctcaatgttctgctcaccctccgtctggtcggtatcaagaaccaaagcctagaacaacagctctcccagcaaatcaaagaggacatcaacagcggcgagatggaactaacttccggcgagcttgcgctcggtatcctcggtctcggtgcttgcgagaatcaagatgaagaattcattcgtggcgctagattggtttcgaagttggaggccaagttccaggcggagatccagaacatggaggcacatgacggtaaccctctcaccaactactaccagctctccctcgcactacttgcattgtgcctcttcaacggcagctatagcgttacttccgtcacctgttatttcacccccgagaacaagaactactatttcggtgaccagttctctgtcgacactggtgccatggccgttctcgccctcacctgcgtacagaagaacacacagaagaacatggacatgaacgccgagggcaagatctcgaactacgttgcttccctcgccaacaagatccaagccgagaggaagcaaggcctattcggcaacatcttctccaccggcgaggccatgcaggcgctgtttgtctcttcggactactataagaacgaggcaaattgccgtgagactttgcgcgctgtgttcgacaacatctcccgcggtgccttctacctgcctatcaccgccgcccagattctgcccgctctcatgggaaagacctacctcgacgtcaccaacccctcctgcggtctcaaccccgtcaagttcaataccagcaccgagaagcctggtaccgtcacgccaactactgctcctctcaacatccttgtcaagtactcggtccgcattaacaagaccagccacaccgaggtcaccgtccgcaagggctccgtcttcttggatgttatgaaggccgcccaggagaaaaacgagaccctcttccgcttcaccgtggaggagacctcctggggtccctacatcacatccgttcagggtatcaaagcttccaataatgaccgcacctactggaagctcctctccaacggccagccactgtcgcaaggtgctggttctcatgtggttcagaacggcgacaacctggaggtccgctggagcaagtactaa

SEQ ID NO: 16 - DNA, 1287 bp, synthetic construct
  source 1..1287: mol_type = other DNA
atgaagttctttgccattgctgccttatttgccgcggctgccgtcgcccagcctaacgtcatctccaagagacagatctgtgtcgtcaacgagaccaacttctggcgtctggagccgttgatcaacaccatgctcaacgccaactacaccaagggtaccctcggtctcaacgtcctcctcaccctccgcctcgtcggtatcaagaaccagtccctcgagcagcagctctcccagcagatcaaggaggacatcaactccggtgagatggagctcacctccggtgagctcgccctcggtatcctcggtctcggtgcctgcgagaaccaggacgaggagttcatccgcggtgcccgcctcgtctccaagctcgaggccaagttccaggccgagatccagaacatggaggcccacgacggtaaccccctcaccaactactaccagctctccctcgccctcctcgccctctgcctcttcaacggttcctactccgtcacctccgtcacctgctacttcacccccgagaacaagaactactacttcggtgaccagttctccgtcgacaccggtgccatggccgtcctcgccctcacctgcgtccagaagaacacccagaagaacatggacatgaacgccgagggtaagatctccaactacgtcgcctccctcgccaacaagatccaggccgagcgcaagcagggtctcttcggtaacatcttctccaccggtgaggccatgcaggccctcttcgtctcctccgactactacaagaacgaggccaactgccgcgagaccctccgcgccgtcttcgacaacatctcccgcggtgccttctacctccccatcaccgccgcccagatcctccccgccctcatgggtaagacctacctcgacgtcaccaacccctcctgcggtctcaaccccgtcaagttcaacacctccaccgagaagcccggtaccgtcacccccaccaccgcccccctcaacatcctcgtcaagtactccgtccgcatcaacaagacctcccacaccgaggtcaccgtccgcaagggttccgtcttcctcgacgtcatgaaggccgcccaggagaagaacgagaccctcttccgcttcaccgtcgaggagacctcctggggtccctacatcacctccgtccagggtatcaaggcctccaacaacgaccgcacctactggaagctcctctccaacggtcagcccctctcccagggtgccggttcccacgtcgtccagaacggtgacaacctcgaggtccgctggtccaagtactaa

SEQ ID NO: 17 - DNA, 1317 bp, synthetic construct
  source 1..1317: mol_type = other DNA
atgaagtttttcgccattgccgccctatttgccgccgctgccgttgcccagcctaacgtcatctcgaagcggaacatctgcgagataaccgaggtggacagcacgctggtggagaggctgggccagcgcctcttgccctggatggaccggctctcccaggagcagctgaaccccagtatctacgtgggcctgcgcctctcgagcctgcaggctggggccaaggaggcccactacctgcacagcctcaagctcagctaccagcagagcctcctgaggcctgcctccaacaaggatgacaatgactccgaggccaagccctctatgggccagctggccctctacctgctggctctccgggccaactgcgagttcatcggaggccgcaagggggacaggctggtctcccagctgaagcggttcctggaggacgagaagagggccatcgggcacaaccaccagggtcacccccgcaccagctactaccagtacagcctgggcatcctggccctgtgtgtccaccagaagcgagtccacgacagtgtggtgggcaagctcctgtacgccgtggaacacaagccgcatctcctgcaggaccacgtctctgtggacaccatggccatggcaggcatggccttctcctgtctggagctgtccaacctcaaccccaagcagagaaaccggatcaacctggccctcaagagagtgcaagagaagatcctgaaggcccagaccccagagggctacttcgggaatgtctacagcacccctctggctttgcagttgctgatgggctccctcaggccctcggtggagctgggcacagcctgccttaaagccaaggctgctctgcaggccagcctacagcacaagaccttccagaaccctctcatgatctctcagctgctgcctgtcctgaaccagaagagctatgtggatctcatctccccagactgccaggctccaagagccctgttggaaccggctctggagaccccgccacaggccaaagtcccgaagttcattgacgtcttgctgaaggtctccggcatctccccttcatacagacactctgtctctgtccctgctggctcctccctggaagacatcctgaagaacgcccaggagcatggaagattcaggtttagaacacaggcctccctgtctggccccttcctgacctccgtgctggggagaaaggctggggaacgtgagttctggcaggtcctccgagatcctgacacccccttgcagcaaggtattgctgactacagacccaaggatggagagaccatcgagctgaggctggttggctggtag

SEQ ID NO: 18 - DNA, 1317 bp, synthetic construct
  source 1..1317: mol_type = other DNA
atgaagttcttcgccattgccgccctatttgccgccgccgccgtcgcccagcccaacgtcatctccaagcgcaacatctgcgagatcaccgaggtcgactccaccctcgtcgagcgcctcggtcagcgcctcctcccctggatggaccgcctctcccaggagcagctcaacccctccatctacgtcggtctccgcctctcctccctccaagccggtgccaaggaggcccactacctccactccctcaagctctcctaccagcagtccctcctccgccccgcctccaacaaggacgacaacgactccgaggccaagccctccatgggtcagctcgccctctacctcctcgccctccgcgccaactgcgagttcatcggtggtcgcaagggtgaccgcctcgtctcccagctcaagcgcttcctcgaggacgagaagcgcgccatcggtcacaaccaccagggtcacccccgcacctcctactaccagtactccctcggtatcctcgccctctgcgtccaccagaagcgcgtccacgactccgtcgtcggtaaactcctctacgccgtcgagcacaagccccacctcctccaagaccacgtctccgtcgacaccatggccatggccggtatggccttctcctgcctcgagctctccaacctcaaccccaagcagcgcaaccgcatcaacctcgccctcaagcgcgtccaagagaagatcctcaaggcccagacccccgagggttacttcggtaacgtctactccacccccctcgccctccaactcctcatgggttccctccgcccctccgtcgagctcggtaccgcctgcctcaaggccaaggccgccctccaagcctccctccaacacaagaccttccaaaaccccctcatgatctcccagctcctccccgtcctcaaccagaagtcctacgtcgacctcatctcccccgactgccaggccccccgcgccctcctcgagcccgccctcgagacccccccccaggccaaggtccccaagttcatcgacgtcctcctcaaggtctccggtatctccccctcctaccgccactccgtctccgtccccgccggttcctccctcgaggacatcctcaagaacgcccaggagcacggtcgcttccgcttccgcacccaggcctccctctccggtcccttcctcacctccgtcctcggtcgcaaggccggtgagcgcgagttctggcaggtcctccgcgaccccgacacccccctccagcagggtatcgccgactaccgccccaaggacggtgagaccatcgagctccgcctcgtcggttggtaa

SEQ ID NO: 19 - DNA, 1317 bp, synthetic construct
  source 1..1317: mol_type = other DNA
atgaagttttttgccattgctgccttatttgccgcggctgccgtcgcccagcctaacgtcatctccaagcgtaacatctgtgagatcactgaagtggactctactctcgttgagcgcttgggtcagcgcctcctcccctggatggaccgcctctcccaggagcagctcaacccctccatctacgtcggtctccgcctctcctccctccaggccggtgccaaggaggcccactacctccactccctcaagctctcctaccagcagtccctcctccgccccgcctccaacaaggacgacaacgactccgaggccaagccctccatgggtcagctcgccctctacctcctcgccctccgcgccaactgcgagttcatcggtggtcgcaagggtgaccgcctcgtctcccagctcaagcgcttcctcgaggacgagaagcgcgccatcggtcacaaccaccagggtcacccccgcacctcctactaccagtactccctcggtatcctcgccctctgcgtccaccagaagcgcgtccacgactccgtcgtcggtaagctcctctacgccgtcgagcacaagccccacctcctccaggaccacgtctccgtcgacaccatggccatggccggtatggccttctcctgcctcgagctctccaacctcaaccccaagcagcgcaaccgcatcaacctcgccctcaagcgcgtccaggagaagatcctcaaggcccagacccccgagggttacttcggtaacgtctactccacccccctcgccctccagctcctcatgggttccctccgcccctccgtcgagctcggtaccgcctgcctcaaggccaaggccgccctccaggcctccctccagcacaagaccttccagaaccccctcatgatctcccagctcctccccgtcctcaaccagaagtcctacgtcgacctcatctcccccgactgccaggccccccgcgccctcctcgagcccgccctcgagacccccccccaggccaaggtccccaagttcatcgacgtcctcctcaaggtctccggtatctccccctcctaccgccactccgtctccgtccccgccggttcctccctcgaggacatcctcaagaacgcccaggagcacggtcgcttccgcttccgcacccaggcctccctctccggtcccttcctcacctccgtcctcggtcgcaaggccggtgagcgcgagttctggcaggtcctccgcgaccccgacacccccctccagcagggtatcgccgactaccgccccaaggacggtgagaccatcgagctccgcctcgtcggttggtaa

SEQ ID NO: 20 - DNA, 1317 bp, synthetic construct
  source 1..1317: mol_type = other DNA
atgaagttttttgccattgctgccttatttgccgcggctgccgtcgcccagcctaacgtcatctccaagcgtaacatctgtgagatcactgaagtggactctactctcgttgagcgcttgggtcaacgcctacttccctggatggacagactctctcaggagcagctgaacccgagcatctatgtcggcctccgtctgtcatccctccaggctggtgctaaggaagcgcactacctccactctctgaaactgtcataccagcagtcgctcctccgccccgcttccaacaaggacgataatgacagcgaggctaaaccatctatgggtcagctagccctctatctgcttgccctgcgggcaaactgcgagttcataggcggaagaaagggtgaccgtctcgtctcccagttgaagagatttctggaagacgaaaagagggctatcggccacaaccaccagggccaccccagaacctcctactaccagtactcgctgggtatcctcgccctctgcgttcatcagaagcgtgtccacgattccgttgtcggtaagctcctctacgcagtggaacacaagccccatttgcttcaagaccacgtgagtgtcgacaccatggccatggcgggtatggcattcagctgcttggagctctccaacctaaaccccaaacagcgtaaccgcatcaacctagcgttgaaacgagtgcaagagaagatccttaaggcccaaacccccgaaggctacttcggtaacgtctactccacacccctcgccctccagctcctcatggggagccttagaccatccgtcgaattaggaaccgcttgcttgaaagcaaaggcagccctgcaagcatctctccagcacaagactttccagaatccactcatgatttctcagctgcttcccgttctcaaccagaagtcgtatgttgacctcattagcccggattgccaggcaccacgagctttacttgagcctgcactggagaccccgccccaggccaaggtccctaagttcatcgatgtgttgctcaaggtcagtggtatcagcccctcctatcgtcactccgtgagcgtgcccgctggaagctctttggaagatatccttaagaatgcccaggagcacggtaggttcaggtttcgtacccaagcctctctttccggccccttcctcaccagcgtacttggccgtaaggccggcgagcgtgagttctggcaggtgctgcgtgatcccgacaccccactgcaacagggcattgcagactaccgaccgaaggatggagagactatcgagctccgccttgtcggttggtaa

SEQ ID NO: 21 - DNA, 1317 bp, synthetic construct
  source 1..1317: mol_type = other DNA
atgaagttctttgccattgctgccttatttgccgcggctgccgtcgcccagcctaacgtcatctccaagcgtaacatctgtgagatcactgaagtggactctactctcgttgagcgcttgggtcaacgcctacttccctggatggacagactctctcaggagcagctgaacccgagcatctatgtcggcctccgtctgtcatccctccaggctggtgctaaggaagcgcactacctccactctctgaaactgtcataccagcagtcgctcctccgccccgcttccaacaaggacgataatgacagcgaggctaaaccatctatgggtcagctagccctctatctgcttgccctgcgggcaaactgcgagttcataggcggaagaaagggtgaccgtctcgtctcccagttgaagagatttctggaagacgaaaagagggctatcggccacaaccaccagggccaccccagaacctcctactaccagtactcgctgggtatcctcgccctctgcgttcatcagaagcgtgtccacgattccgttgtcggtaagctcctctacgcagtggaacacaagccccatttgcttcaagaccacgtgagtgtcgacaccatggccatggcgggtatggcattcagctgcttggagctctccaacctaaaccccaaacagcgtaaccgcatcaacctagcgttgaaacgagtgcaagagaagatccttaaggcccaaacccccgaaggctacttcggtaacgtctactccacacccctcgccctccagctcctcatggggagccttagaccatccgtcgaattaggaaccgcttgcttgaaagcaaaggcagccctgcaagcatctctccagcacaagactttccagaatccactcatgatttctcagctgcttcccgttctcaaccagaagtcgtatgttgacctcattagcccggattgccaggcaccacgagctttacttgagcctgcactggagaccccgccccaggccaaggtccctaagttcatcgatgtgttgctcaaggtcagtggtatcagcccctcctatcgtcactccgtgagcgtgcccgctggaagctctttggaagatatccttaagaatgcccaggagcacggtaggttcaggtttcgtacccaagcctctctttccggccccttcctcaccagcgtacttggccgtaaggccggcgagcgtgagttctggcaggtgctgcgtgatcccgacaccccactgcaacagggcattgcagactaccgaccgaaggatggagagactatcgagctccgccttgtcggttggtaa

SEQ ID NO: 22 - DNA, 1317 bp, synthetic construct
  source 1..1317: mol_type = other DNA
atgaagttctttgccattgctgccttatttgccgcggctgccgtcgcccagcctaacgtcatctccaagcgtaacatctgtgagatcactgaagtggactctactctcgttgagcgcttgggtcagcgcctcctcccctggatggaccgcctctcccaggagcagctcaacccctccatctacgtcggtctccgcctctcctccctccaggccggtgccaaggaggcccactacctccactccctcaagctctcctaccagcagtccctcctccgccccgcctccaacaaggacgacaacgactccgaggccaagccctccatgggtcagctcgccctctacctcctcgccctccgcgccaactgcgagttcatcggtggtcgcaagggtgaccgcctcgtctcccagctcaagcgcttcctcgaggacgagaagcgcgccatcggtcacaaccaccagggtcacccccgcacctcctactaccagtactccctcggtatcctcgccctctgcgtccaccagaagcgcgtccacgactccgtcgtcggtaagctcctctacgccgtcgagcacaagccccacctcctccaggaccacgtctccgtcgacaccatggccatggccggtatggccttctcctgcctcgagctctccaacctcaaccccaagcagcgcaaccgcatcaacctcgccctcaagcgcgtccaggagaagatcctcaaggcccagacccccgagggttacttcggtaacgtctactccacccccctcgccctccagctcctcatgggttccctccgcccctccgtcgagctcggtaccgcctgcctcaaggccaaggccgccctccaggcctccctccagcacaagaccttccagaaccccctcatgatctcccagctcctccccgtcctcaaccagaagtcctacgtcgacctcatctcccccgactgccaggccccccgcgccctcctcgagcccgccctcgagacccccccccaggccaaggtccccaagttcatcgacgtcctcctcaaggtctccggtatctccccctcctaccgccactccgtctccgtccccgccggttcctccctcgaggacatcctcaagaacgcccaggagcacggtcgcttccgcttccgcacccaggcctccctctccggtcccttcctcacctccgtcctcggtcgcaaggccggtgagcgcgagttctggcaggtcctccgcgaccccgacacccccctccagcagggtatcgccgactaccgccccaaggacggtgagaccatcgagctccgcctcgtcggttggtaa

SEQ ID NO: 23 - DNA, 116 bp, synthetic construct
  source 1..116: mol_type = other DNA
gttcaaagactaggatggataaatggggtatataaagcaccctgactcccttcctccaagttctatctaaccagccatcctacactctacatatccacaccaatctactagtcaac

SEQ ID NO: 24 - DNA, 48 bp, synthetic construct
  source 1..48: mol_type = other DNA
atgaagtttttcgccattgccgccctatttgccgccgctgccgttgcc

SEQ ID NO: 25 - DNA, 63 bp, synthetic construct
  source 1..63: mol_type = other DNA
atgatggtcgcttggtggagtttgtttttgtatgggctacaggtagctgcgccggcactagca

SEQ ID NO: 26 - DNA, 78 bp, synthetic construct
  source 1..78: mol_type = other DNA
atgcgtaataatctcttattttcattaaatgccatcgctggagcagtagcgcacccgtcttttccgatccataagcga

SEQ ID NO: 27 - DNA, 54 bp, synthetic construct
  source 1..54: mol_type = other DNA
atgccttgggccggtttagccacactttttctagcagttgcgggcgtacacggg

SEQ ID NO: 28 - DNA, 51 bp, synthetic construct
  source 1..51: mol_type = other DNA
atgcagttttccgtcgccgccgtcctcgctctagctaccgcagtcgccgcg

SEQ ID NO: 29 - DNA, 78 bp, synthetic construct
  source 1..78: mol_type = other DNA
atgtctcacgtcgaacacttacgccacactgcactgcaactaaggagtcccctatttacctgcccccatttacctgta

SEQ ID NO: 30 - DNA, 60 bp, synthetic construct
  source 1..60: mol_type = other DNA
atgactccttctttatctaaactcgttgcccttagcttattcctcgggactgccttaggg

SEQ ID NO: 31 - DNA, 63 bp, synthetic construct
  source 1..63: mol_type = other DNA
atggccctccctcttcttgtcaacgctggtttattggctttaccgatcgcaggtagcatcggc

SEQ ID NO: 32 - DNA, 78 bp, synthetic construct
  source 1..78: mol_type = other DNA
atggagaaaggagggtcagtatttagtagcagggtatgggggattaagcgaggatctaatagtaggtttttcttcggg

SEQ ID NO: 33 - protein, 21 aa, synthetic construct
MMVAWWSLFLYGLQVAAPALA

SEQ ID NO: 34 - protein, 26 aa, synthetic construct
MRNNLLFSLNAIAGAVAHPSFPIHKR

SEQ ID NO: 35 - protein, 18 aa, synthetic construct
MPWAGLATLFLAVAGVHG

SEQ ID NO: 36 - protein, 17 aa, synthetic construct
MQFSVAAVLALATAVAA

SEQ ID NO: 37 - protein, 26 aa, synthetic construct
MSHVEHLRHTALQLRSPLFTCPHLPV

SEQ ID NO: 38 - protein, 20 aa, synthetic construct
MTPSLSKLVALSLFLGTALG

SEQ ID NO: 39 - protein, 21 aa, synthetic construct
MALPLLVNAGLLALPIAGSIG

SEQ ID NO: 40 - protein, 26 aa, synthetic construct
MEKGGSVFSSRVWGIKRGSNSRFFFG

(19) United States
(12) Patent Application Publication
(10) Pub. No.: US 2024/0407381 A1
(43) Pub. Date: Dec. 12, 2024

(54) FOOD COMPOSITIONS COMPRISING RECOMBINANT CELLS COMPRISING TRANSCOBALAMIN

(71) Applicant: IMAGINDAIRY LTD., Ashdod (IL)

(72) Inventors: Arie ABO, Emeryville, CA (US); Ariella LAVY-ALPEROVITCH, Haifa (IL)

(21) Appl. No.: 18/701,958

(22) PCT Filed: Nov. 17, 2022
(86) PCT No.: PCT/IL2022/051230

§ 371 Date: Apr. 17, 2024

Related U.S. Application Data
(60) Provisional application No. 63/280,739, filed on Nov. 18, 2021.

(51) Int. Cl. A23C 11/06; A23J 1/00; A23J 3/08; A23J 3/20; C07K 14/47; C12N 1/14

(52) U.S. Cl. CPC A23C 11/065; A23J 1/008; A23J 3/08; A23J 3/20; C07K 14/47; C12N 1/14

(57) ABSTRACT

The present invention provides a composition comprising cobalamin and a mammalian transcobalamin (TC) polypeptide, heterologously expressed in a recombinant cell. Further provided are food compositions comprising the cobalamin bound to the recombinant TC polypeptide, as well as methods of producing thereof, such as producing a dairy-like food composition.

### CROSS REFERENCE TO RELATED APPLICATIONS

This application claims the benefit of priority of U.S. Provisional Patent Application No. 63/280,739, filed Nov. 18, 2021, entitled “FOOD COMPOSITIONS COMPRISING RECOMBINANT CELLS COMPRISING TRANSCOBALAMIN”, the contents of which are all incorporated by reference in their entirety.

### FIELD OF INVENTION

The present invention is in the field of producing dairy-like compositions.

### BACKGROUND

Cobalamin or vitamin B₁₂ is a water-soluble vitamin which forms part of the vitamin B complex found in foods. Cobalamin is the only vitamin which cannot be synthesized by animals or plants and must be absorbed from food in the gut. It is synthesized by micro-organisms, in particular by anaerobic bacteria and yeasts.

Cobalamin does not circulate in the body in a free form in an appreciable amount and is bound by transcobalamin (as well as albumin).

Without transcobalamin (TC), cobalamin cannot cross cell membranes. TC is a single chain polypeptide of 45 kDa found primarily in serum, seminal fluid and cerebro-spinal fluid. Cobalamin bound TC or holo-TC, attaches to specific receptors on cell membranes and once bound, the holo-TC complex is taken into cells by pinocytosis. Cobalamin bound TC or holo-TC, attaches to specific receptors on cell membranes and once bound, the holo-TC complex is taken into cells by pinocytosis. Transcobalamin I (TCN1), is a glycoprotein produced by the salivary glands, and mainly serves to protect cobalamin from acid degradation in the stomach. Transcobalamin II (TCN2) is known to bind cobalamin once it has been taken up by enterocytes of the terminal ileum and is involved in the transport of cobalamin to the tissues.

In order to make an economical synthetic substitute for milk it is necessary to synthesize efficiently TC in various host cells and to produce food compositions that contain TC-B12 complexes. Improved expression systems (e.g., recombinant cells) and plasmids, for the production of a TC protein and cobalamin, are greatly needed.

### SUMMARY

According to a first aspect there is provided a composition comprising cobalamin and a recombinant cell comprising an exogenous polynucleotide comprising a nucleic acid sequence encoding a mammalian transcobalamin (TC) polypeptide or a material derived or processed from the recombinant cell.

In some embodiments, the recombinant cell is any one of: a unicellular organism, a cell of a multicellular organism, and a cell in a culture.

In some embodiments, the unicellular organism is a bacterium or a fungus.

In some embodiments, the fungus is selected from the group consisting of: Aspergillus oryzae and Trichoderma reesei.

In some embodiments, the recombinant cell is Aspergillus oryzae.

In some embodiments, the TC polypeptide comprises: transcobalamin 1 (haptocorrin), transcobalamin 2, or both.

In some embodiments, the material derived or processed from the recombinant cell comprises any one of: cell secretome, cell lysate, cell extract, cell homogenate, a portion thereof, a fraction thereof, and any combination thereof, of the recombinant cell.

In some embodiments, the mammalian TC comprises an amino acid sequence having at least 70% homology to an amino acid sequence set forth in any one of SEQ ID Nos: 3-6.

In some embodiments, the TC polypeptide is bound to the cobalamin.

In some embodiments, the exogenous polynucleotide encoding the mammalian TC polypeptide is codon optimized for expression in the recombinant cell.

In some embodiments, the mammalian TC further comprises a signal peptide comprising an amino acid sequence having at least 70% homology to an amino acid sequence set forth in any one of SEQ ID Nos: 7, 8, and 33-40.

In some embodiments, the exogenous polynucleotide comprises a nucleic acid sequence having at least 70% identity to a nucleic acid sequence set forth in any one of SEQ ID Nos: 1, 2, and 9-22.

In some embodiments, the exogenous polynucleotide further comprises a nucleic acid sequence encoding a signal peptide, wherein the exogenous polynucleotide comprises a nucleic acid sequence having at least 70% identity to a nucleic acid sequence set forth in any one of SEQ ID Nos: 24-32.

In some embodiments, the composition further comprises a nutraceutically acceptable carrier.

In some embodiments, the composition is a food composition.

In some embodiments, the composition is a dairy composition.

In some embodiments, the composition further comprising a milk protein or a plurality thereof, being derived from a fungal cell, a bacterial cell, or any combination thereof.

In some embodiments, the food composition is selected from the group consisting of: a milk, a yogurt, a cheese, a butter, a caseinate, a cream, an infant formula, an ice cream, a frozen custard, a cottage cheese, a cream cheese, a crème fraiche, and a curd.

According to another aspect, there is provided a method for producing a composition comprising a TC-cobalamin complex, the method comprising: (a) culturing a recombinant cell comprising an exogenous polynucleotide comprising a nucleic acid sequence encoding a mammalian TC polypeptide; (b) culturing the cell from step (a) such that the mammalian TC polypeptide encoded from the exogenous polynucleotide is expressed; and; (c) contacting the mammalian TC with cobalamin, thereby producing the composition comprising a TC-cobalamin complex.

In some embodiments, the recombinant cell further comprises the cobalamin.

In some embodiments, the recombinant cell is a fungal cell or a bacterial cell.

In some embodiments, the method further comprising a step preceding step (a) comprising introducing or transfecting a cell with the exogenous polynucleotide, thereby obtaining the recombinant cell.

In some embodiments, the method further comprising a step after step (b) and before step (c), comprising: a) separating the cultured cell from a medium wherein the cell is cultured; and, b) extracting any one of: i. the separated medium, ii. the separated cell; and, iii. both (i) and (ii).

In some embodiments, the method further comprising a step preceding step (c) comprising isolating the TC polypeptide from the extract.

In some embodiments, the method further comprising a step (d) comprising mixing the TC-cobalamin complex with a milk protein or plurality thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes an image of polymerase chain reaction (PCR) of open reading frame (ORF) verifying genomic DNA of transcobalamin 2 (TC2).

FIG. 2 includes an image of western blot analysis demonstrating the expression of TC2. The membranes were incubated with rabbit anti TC2 antibody (1:1000) and anti-rabbit secondary antibody (1:10000), to specifically detect TC2. A band suggestive for TC2 expression, in a size of about 47 kDa, appeared in sample numbers 13 and 16.

FIG. 3 includes an image of the samples that were examined by proteomics analysis. Samples were stained by Coomassie blue after separation by 12% SDS-PAGE. The samples consisted of either merely the secreted proteins, named “secreted”, or both the secreted proteins as well as the proteins extracted from A. orizae cells (named “total”).

FIG. 4 includes an illustrating image of the amino acid sequence of the expressed TC2 in A. orizae, as determined by mass spectrometric proteomic analysis. The signal peptide comprises 18 residues. TC2 was identified in its secreted form. Two additional isoforms were found: a protein that starts at position 22 and a protein that starts at position 25, from the N terminus, indicating 2 additional N-terminal cleavage sites. N-glyco motifs are also demonstrated.

### DETAILED DESCRIPTION

### Cells

According to some embodiments, there is provided a cell comprising a polynucleotide encoding a nucleic acid sequence encoding transcobalamin. In some embodiments, the cell comprises cobalamin (vitamin B12). In some embodiments, the cell comprises a nucleic acid sequence encoding transcobalamin and cobalamin. In some embodiments, the cell comprises a nucleic acid sequence encoding transcobalamin and/or a polypeptide product thereof (hereinafter, transcobalamin polypeptide). In some embodiments, the cell comprises a transcobalamin polypeptide and cobalamin. In some embodiments, the cell comprises a nucleic acid sequence encoding transcobalamin, a transcobalamin polypeptide, and cobalamin. In some embodiments, the cell comprises transcobalamin polypeptide being bound to cobalamin (transcobalamin-cobalamin complex). In some embodiments the cell comprises a heterologous nucleic acid encoding transcobalamin. In some embodiments, the cell does not comprise or produce cobalamin. In some embodiments, the transcobalamin, expressed by the recombinant cell, is being bound to cobalamin outside the recombinant cell. In some embodiments, transcobalamin, extracted from the cell, from the cell secreted medium, or both, is being bound to cobalamin, to form a transcobalamin-cobalamin complex. In some embodiments, cobalamin and transcobalamin polypeptide are derived from the same recombinant cell. In some embodiments, the transcobalamin polypeptide and cobalamin are not derived from the same cell. In some embodiments, transcobalamin polypeptide is expressed in the recombinant cell of the invention, and cobalamin is derived from another cell, that naturally produces cobalamin. In some embodiments, cobalamin is derived from a genetically modified cell, in which the genetic modification shifts the cell from a non-cobalamin producer to a cobalamin producer. As used herein, the term “recombinant polypeptide” encompasses a polypeptide derived from its heterologous expression in a recombinant cell.

In some embodiments, the transcobalamin is a mammalian transcobalamin. In some embodiments, the transcobalamin is bovine transcobalamin. In some embodiments, the mammalian transcobalamin is bovine transcobalamin.

In some embodiments, the transcobalamin is transcobalamin 1, e.g., haptocorrin, R-factor, and R-protein. In some embodiments, the transcobalamin is transcobalamin 2.

In some embodiments, the transcobalamin is a plurality of transcobalamin polypeptides, comprising transcobalamin 1 (i.e., haptocorrin), and transcobalamin 2.

As used herein, the term “transcobalamin” refers to any peptide, polypeptide, or a protein, capable of binding cobalamin, e.g., vitamin B12, protecting cobalamin from acid degradation, transporting cobalamin from enterocytes to peripheral tissues, or any combination thereof.

In some embodiments, the cell is a recombinant cell. In some embodiments, the cell is a transformed cell. In some embodiments, the cell is a transgenic cell.

In some embodiments, the cell is selected from: a unicellular organism, a cell of a multicellular organism, and a cell in a culture.

In some embodiments, the cell is a prokaryote cell or a eukaryote cell.

In some embodiment, the cell is a unicellular organism.

In some embodiments, a unicellular organism is a bacterium or a fungus. In some embodiments, the unicellular organism is a bacterium. In some embodiments, the fungus is a yeast cell. In some embodiments, the fungus is a filamentous fungus.

In some embodiments, the bacterium belongs to a genus selected from: Bacillus, Pseudomonas, or Propionibacterium. In some embodiments, the bacterium comprises a plurality of bacteria genera comprising any combination selected from: Bacillus, Pseudomonas, and Propionibacterium.

In some embodiments, the bacterium or a plurality thereof is Bacillus megaterium, Pseudomonas denitrificans, Propionibacterium freudenreichii, or any combination thereof.

In some embodiments, the bacterium is a Bacillus megaterium. In some embodiments, the cell of the invention is Bacillus megaterium. In some embodiments, the recombinant cell of the invention, comprising an exogenous polynucleotide encoding a transcobalamin polypeptide and cobalamin is Bacillus megaterium.

In some embodiments, there is provided a recombinant Bacillus megaterium cell comprising: (i) an exogenous polynucleotide comprising a nucleic acid sequence encoding a mammalian transcobalamin polypeptide, and (ii) cobalamin.

In some embodiments, the recombinant cell is a fungal cell. In some embodiments, the recombinant cell is a yeast. In some embodiments the fungal cell comprises a filamentous fungus. In some embodiments, the fungal cell is Aspergillus orizae (A. orizae), Trichoderma reesei, or both. In some embodiments, the cell of the invention comprises A. orizae. In some embodiments, there is provided a recombinant A. orizae cell comprising an exogenous polynucleotide comprising a nucleic acid sequence encoding a mammalian transcobalamin polypeptide.

In some embodiments, a mammalian comprises livestock.

As used herein, the term “livestock” comprises any domestic mammal, semi-domestic mammal or captive wild mammal. Non-limiting examples of non-human mammals include: antelope, bear, beaver, bison, boar, camel, caribou, cattle, deer, elephant, elk, fox, giraffe, goat, hare, horse, ibex, kangaroo, lion, llama, moose, peccary, pig, rabbit, seal, sheep, squirrel, tiger, whale, yak, and zebra, or combinations thereof.

In some embodiments, a livestock is selected form: cattle, sheep, goat, horse, or swine. In some embodiments, a livestock is or comprises cattle. In some embodiments, a livestock is bovine.

According to some embodiments, there is provided a transgenic cell comprising: (a) the DNA molecule disclosed herein; (b) the artificial nucleic acid molecule disclosed herein; (c) the plasmid or agrobacterium disclosed herein; (d) the protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

### Polynucleotides and Protein Products

In some embodiments, the cell of the invention, as disclosed herein, comprises an exogenous polynucleotide comprising a nucleic acid sequence encoding the transcobalamin polypeptide.

As used herein, the term “exogenous” refers to any polynucleotide, e.g., DNA, which originates or is derived from a source being outside a cell, as used herein. In some embodiments, the exogenous polynucleotide is present in a cell as disclosed herein when being a recombinant, transformed, transfected, transgenic cell. In some embodiments, a wildtype or an intact cell or a genetic background cell of the recombinant cell of the invention, as disclosed herein, is devoid of or lacks the exogenous polynucleotide as disclosed herein.

In some embodiments, the polynucleotide comprises DNA, RNA, or any hybrid thereof. In some embodiments, the terms “polynucleotide” and “DNA molecule” are used herein interchangeably.

In some embodiments, the DNA molecule is a polynucleotide. In some embodiments, the DNA molecule is an isolated DNA molecule or an isolated polynucleotide. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises, consists of, or derived from the nucleic acid sequence:

(SEQ ID NO: 1)
CACGGCTGAAGAGATGAGACCATCAGGCCAGCTCCCCCTAACGGGGCTGC
TATTCTTTTCTCTTATTCCCAGTCAACTATGCCAGATCTGTGTGGTAAAT
GAAACGAACTTCTGGCGTCTAGAACCTCTGATAAACACGATGCTGAATGC
AAACTACACCAAAGGGACCCTAGGTCTCAATGTCCTGTTGACCTTGAGAC
TTGTTGGGATTAAGAATCAAAGCCTAGAACAACAGCTGAGTCAACAAATC
AAAGAAGACATAAACAGCGGAGAGATGGAGTTAACCTCGGGAGAGCTTGC
CTTGGGTATTCTGGGTTTGGGAGCATGTGAAAATCAGGATGAAGAGTTTA
TACGTGGTGCTCGTCTGGTTAGCAAACTTGAAGCCAAATTCCAAGCAGAA
ATTCAGAACATGGAGGCACATGACGGCAACCCACTGACCAACTACTACCA
GCTCAGCCTGGCCCTGCTGGCCTTGTGTCTGTTCAACGGCAGCTATTCAG
TCACCAGTGTTACCTGTTACTTCACTCCTGAAAATAAGAACTATTATTTT
GGGGACCAGTTCTCAGTAGATACTGGAGCGATGGCTGTTCTTGCTTTAAC
ATGTGTGCAGAAGAATACACAAAAGAATATGGACATGAACGCAGAGGGTA
AAATCAGTAATTACGTGGCGTCACTGGCAAACAAGATCCAGGCTGAGAGA
AAACAGGGTCTCTTTGGAAACATATTTAGCACGGGAGAAGCTATGCAGGC
TCTCTTTGTCTCATCGGACTACTACAAAAATGAAGCAAATTGCAGAGAAA
CTCTACGTGCAGTATTTGACAACATTTCTCGGGGAGCATTTTACTTGCCG
ATTACTGCAGCCCAGATCTTACCTGCCTTGATGGGAAAGACCTATCTGGA
TGTCACCAACCCTTCTTGTGGCCTCAATCCAGTCAAATTCAACACCTCCA
CTGAAAAGCCTGGTACTGTGACACCTACAACCGCACCCTTGAATATCTTA
GTCAAATACTCTGTGCGAATCAACAAGACAAGTCACACTGAAGTCACTGT
GAGAAAGGGTTCTGTCTTCCTCGACGTGATGAAGGCTGCTCAGGAGAAAA
ATGAGACCCTATTTCGCTTCACAGTGGAGGAGACCTCGTGGGGCCCCTAC
ATCACCTCTGTTCAGGGCATAAAGGCCAGCAATAATGACAGAACCTACTG
GAAGCTTCTGAGCAACGGCCAGCCACTGAGCCAAGGAGCTGGGAGTCATG
TTGTCCAAAATGGAGACAATTTGGAGGTTCGTTGGAGCAAATACTAATAA
ATTCAAACCTTCCTCAGCTGGGTCTGAGCCTTTTGTGGTACAGTTCTATG
TGGAAATTGATCTTACATGTTCCTTTTCATTTATATCTCAATCCTCACAG
CACATCAATAACCTCTACTTCTCTCTTTGAGTGTCCAATAAAAGACCAAC
AGC.

In some embodiments, the polynucleotide comprises a nucleic acid sequence with at least 70%, at least 80%, at least 90%, at least 95%, at least 97%, at least 99%, or 100% homology or identity to SEQ ID NO: 1, or any value and range therebetween. Each possibility represents a separate embodiment of the invention. In some embodiments, the polynucleotide comprises a nucleic acid sequence with 70% to 100% homology or identity to SEQ ID NO: 1. Each possibility represents a separate embodiment of the invention.

In some embodiments, the polynucleotide encoding transcobalamin 1 comprises or consists of a nucleic acid sequence set forth in SEQ ID NO: 1.

In some embodiments, the polynucleotide comprises, consists of, or derived from the nucleic acid sequence:

(SEQ ID NO: 2)
CTGTCCCAGGCCGGGGTGGGCAGTTTCAGGGGCTCCTTCTGGGAGGGGTA
GCAGGTAGGCACCCTAGCCTACCGTGTCTACCGAGGGCATACAGACAGGC
GTGGCTGGACAGACTCAGCAGTCAGACGTTCTGCACCCCCATCCAGGGCT
CTTCGGGCGGCGGGGAAAGTGTGGCTGGCCGCACTCGCCCCTGCCCCCGC
CTCCAGGAAGGCCCGCGCTGCTCTGCCCTTCCTTCCCCTGTTCTTTGGCC
TCCCTTTCCCCTCAGCTACAGACCTTTTCCCTTTTCCCTCTGCTGACGTA
GCAATGTGCTGCCGGGCGTGGAGGGATTAATCAGTGACAGGAAGCTGCCT
CTTTCGGAGCAGTGACCACTGTGGTCAGGAGAGCCTCTCTCTACCCGGCC
AGCCCTAGGAGCCTGTCCCGCTCCCCGCCTGCTGTCCACCCCCTGCTCCT
GCCATGGGGCACCTCGGGGCCCTCCTCTTCCTGCTGGGGGGCCTGGGAGC
GCTAGCCAACATCTGCGAGATAACCGAGGTGGACAGCACGCTGGTGGAGA
GGCTGGGCCAGCGCCTCTTGCCCTGGATGGACCGGCTCTCCCCGGAGCAG
CTGAACCCCAGTATCTACGTGGGCCTGCGCCTCTCGAGCCTGCAGGCTGG
GGCCAAGGAGGCCCACTACCTGCACAGCCTCAAGCTCAGCTACCAGCAGA
GCCTCCTGAGGCCTGCCTCCAACAAGGATGACAATGACTCCGAGGCCAAG
CCCTCTATGGGCCAGCTGGCCCTCTACCTGCTGGCTCTCCGGGCCAACTG
CGAGTTCATCGGAGGCCGCAAGGGGGACAGGCTGGTCTCCCAGCTGAAGC
GGTTCCTGGAGGACGAGAAGAGGGCCATCGGGCACAACCACCAGGGTCAC
CCCCGCACCAGCTACTACCAGTACAGCCTGGGCATCCTGGCCCTGTGTGT
CCACCAGAAGCGAGTCCACGACAGTGTGGTGGGCAAGCTCCTGTACGCCG
TGGAACACAAGCCGCATCTCCTGCAGGACCACGTCTCTGTGGACACCATG
GCCATGGCAGGCATGGCCTTCTCCTGTCTGGAGCTGTCCAACCTCAACCC
CAAGCAGAGAAACCGGATCAACCTGGCCCTCAAGAGAGTGCAAGAGAAGA
TCCTGAAGGCCCAGACCCCAGAGGGCTACTTCGGGAATGTCTACAGCACC
CCTCTGGCTTTGCAGTTGCTGATGGGCTCCCTCAGGCCCTCGGTGGAGCT
GGGCACAGCCTGCCTTAAAGCCAAGGCTGCTCTGCAGGCCAGCCTACAGC
ACAAGACCTTCCAGAACCCTCTCATGATCTCTCAGCTGCTGCCTGTCCTG
AACCAGAAGAGCTATGTGGATCTCATCTCCCCAGACTGCCAGGCTCCAAG
AGCCCTGTTGGAACCGGCTCTGGAGACCCCGCCACAGGCCAAAGTCCCGA
AGTTCATTGACGTCTTGCTGAAGGTCTCCGGCATCTCCCCTTCATACAGA
CACTCTGTCTCTGTCCCTGCTGGCTCCTCCCTGGAAGACATCCTGAAGAA
CGCCCAGGAGCATGGAAGATTCAGGTTTAGAACACAGGCCTCCCTGTCTG
GCCCCTTCCTGACCTCCGTGCTGGGGAGAAAGGCTGGGGAACGTGAGTTC
TGGCAGGTCCTCCGAGATCCTGACACCCCCTTGCAGCAAGGTATTGCTGA
CTACAGACCCAAGGATGGAGAGACCATCGAGCTGAGGCTGGTTGGCTGGT
AGCCCCCCAGGCTCACCCACGCCGCTGCCTCCCACACTTCCTGTCTCCCA
CCCTCCCTCCAGACACCCCGGTAATGGGCGCACGCCTGATCCTGCTGCCT
TCTCTTACACACTTGTCCCTGGATCCCACAGCCGCCAGCCCTGCGGGAGT
CCTAAACCACCGCCCACCTGGAGCAGGGAGCCCAGCACCTCCCCGAGGAG
GTCTGTCTGCCTGGGTCTGGTCCCACCTGGCCCGGCGGGTGTCCCGAGAA
GGTCACTCACAGGCCGGAGGGCGTAAACGTCTCGGACTCCTCCCCGGCCG
CCTCGTGAGTACCGCTCGCTCTGCGATGGGGGTCGCTTGAGGACAGCCCT
GCGGCCCTGACCCTAGCTCTCCACTCTGTCGTCCAGGCTGCCGCCCCAGA
GCCGGCTGCGTTGTAGCGGCTGGTGAGGGTTTCTGCAGAGCTCTCAGCTC
ACCTCTAGAAGGATTAAAGAGTTGGTTGTGCACGTG.

In some embodiments, the polynucleotide comprises a nucleic acid sequence with at least 70%, at least 80%, at least 90%, at least 95%, at least 97%, at least 99%, or 100% homology or identity to SEQ ID NO: 2, or any value and range therebetween. Each possibility represents a separate embodiment of the invention. In some embodiments, the polynucleotide comprises a nucleic acid sequence with 70% to 100% homology or identity to SEQ ID NO: 2. Each possibility represents a separate embodiment of the invention.

In some embodiments, the polynucleotide encoding transcobalamin 2 comprises or consists of a nucleic acid sequence set forth in SEQ ID NO: 2.

In some embodiments, the polynucleotide comprises or consists the nucleic acid sequence:

(SEQ ID NO: 9)
CAGATCTGTGTGGTAAATGAAACGAACTTCTGGCGTCTAGAACCTCTGAT
AAACACGATGCTGAATGCAAACTACACCAAAGGGACCCTAGGTCTCAATG
TCCTGTTGACCTTGAGACTTGTTGGGATTAAGAATCAAAGCCTAGAACAA
CAGCTGAGTCAACAAATCAAAGAAGACATAAACAGCGGAGAGATGGAGTT
AACCTCGGGAGAGCTTGCCTTGGGTATTCTGGGTTTGGGAGCATGTGAAA
ATCAGGATGAAGAGTTTATACGTGGTGCTCGTCTGGTTAGCAAACTTGAA
GCCAAATTCCAAGCAGAAATTCAGAACATGGAGGCACATGACGGCAACCC
ACTGACCAACTACTACCAGCTCAGCCTGGCCCTGCTGGCCTTGTGTCTGT
TCAACGGCAGCTATTCAGTCACCAGTGTTACCTGTTACTTCACTCCTGAA
AATAAGAACTATTATTTTGGGGACCAGTTCTCAGTAGATACTGGAGCGAT
GGCTGTTCTTGCTTTAACATGTGTGCAGAAGAATACACAAAAGAATATGG
ACATGAACGCAGAGGGTAAAATCAGTAATTACGTGGCGTCACTGGCAAAC
AAGATCCAGGCTGAGAGAAAACAGGGTCTCTTTGGAAACATATTTAGCAC
GGGAGAAGCTATGCAGGCTCTCTTTGTCTCATCGGACTACTACAAAAATG
AAGCAAATTGCAGAGAAACTCTACGTGCAGTATTTGACAACATTTCTCGG
GGAGCATTTTACTTGCCGATTACTGCAGCCCAGATCTTACCTGCCTTGAT
GGGAAAGACCTATCTGGATGTCACCAACCCTTCTTGTGGCCTCAATCCAG
TCAAATTCAACACCTCCACTGAAAAGCCTGGTACTGTGACACCTACAACC
GCACCCTTGAATATCTTAGTCAAATACTCTGTGCGAATCAACAAGACAAG
TCACACTGAAGTCACTGTGAGAAAGGGTTCTGTCTTCCTCGACGTGATGA
AGGCTGCTCAGGAGAAAAATGAGACCCTATTTCGCTTCACAGTGGAGGAG
ACCTCGTGGGGCCCCTACATCACCTCTGTTCAGGGCATAAAGGCCAGCAA
TAATGACAGAACCTACTGGAAGCTTCTGAGCAACGGCCAGCCACTGAGCC
AAGGAGCTGGGAGTCATGTTGTCCAAAATGGAGACAATTTGGAGGTTCGT
TGGAGCAAATACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 9, or any value and range therebetween. Each possibility represents a separate embodiment of the invention. In some embodiments, the polynucleotide comprises a nucleic acid sequence with 70% to 100% homology or identity to SEQ ID NO: 9. Each possibility represents a separate embodiment of the invention.

In some embodiments, the polynucleotide encoding transcobalamin 1 comprises or consists of a nucleic acid sequence set forth in SEQ ID NO: 9. In some embodiments, the polynucleotide further comprises a nucleic acid sequence encoding a signal or a leading peptide. In some embodiments, the signal peptide is directing the transcobalamin polypeptide for secretion from the cell.

In some embodiments, the polynucleotide comprises or consists of the nucleic acid sequence:

(SEQ ID NO: 10)
AACATCTGCGAGATAACCGAGGTGGACAGCACGCTGGTGGAGAGGCTGGG
CCAGCGCCTCTTGCCCTGGATGGACCGGCTCTCCCAGGAGCAGCTGAACC
CCAGTATCTACGTGGGCCTGCGCCTCTCGAGCCTGCAGGCTGGGGCCAAG
GAGGCCCACTACCTGCACAGCCTCAAGCTCAGCTACCAGCAGAGCCTCCT
GAGGCCTGCCTCCAACAAGGATGACAATGACTCCGAGGCCAAGCCCTCTA
TGGGCCAGCTGGCCCTCTACCTGCTGGCTCTCCGGGCCAACTGCGAGTTC
ATCGGAGGCCGCAAGGGGGACAGGCTGGTCTCCCAGCTGAAGCGGTTCCT
GGAGGACGAGAAGAGGGCCATCGGGCACAACCACCAGGGTCACCCCCGCA
CCAGCTACTACCAGTACAGCCTGGGCATCCTGGCCCTGTGTGTCCACCAG
AAGCGAGTCCACGACAGTGTGGTGGGCAAGCTCCTGTACGCCGTGGAACA
CAAGCCGCATCTCCTGCAGGACCACGTCTCTGTGGACACCATGGCCATGG
CAGGCATGGCCTTCTCCTGTCTGGAGCTGTCCAACCTCAACCCCAAGCAG
AGAAACCGGATCAACCTGGCCCTCAAGAGAGTGCAAGAGAAGATCCTGAA
GGCCCAGACCCCAGAGGGCTACTTCGGGAATGTCTACAGCACCCCTCTGG
CTTTGCAGTTGCTGATGGGCTCCCTCAGGCCCTCGGTGGAGCTGGGCACA
GCCTGCCTTAAAGCCAAGGCTGCTCTGCAGGCCAGCCTACAGCACAAGAC
CTTCCAGAACCCTCTCATGATCTCTCAGCTGCTGCCTGTCCTGAACCAGA
AGAGCTATGTGGATCTCATCTCCCCAGACTGCCAGGCTCCAAGAGCCCTG
TTGGAACCGGCTCTGGAGACCCCGCCACAGGCCAAAGTCCCGAAGTTCAT
TGACGTCTTGCTGAAGGTCTCCGGCATCTCCCCTTCATACAGACACTCTG
TCTCTGTCCCTGCTGGCTCCTCCCTGGAAGACATCCTGAAGAACGCCCAG
GAGCATGGAAGATTCAGGTTTAGAACACAGGCCTCCCTGTCTGGCCCCTT
CCTGACCTCCGTGCTGGGGAGAAAGGCTGGGGAACGTGAGTTCTGGCAGG
TCCTCCGAGATCCTGACACCCCCTTGCAGCAAGGTATTGCTGACTACAGA
CCCAAGGATGGAGAGACCATCGAGCTGAGGCTGGTTGGCTGGTAG.

In some embodiments, the polynucleotide comprises a nucleic acid sequence with at least 70%, at least 80%, at least 90%, at least 95%, at least 97%, at least 99%, or 100% homology or identity to SEQ ID NO: 10, or any value and range therebetween. Each possibility represents a separate embodiment of the invention. In some embodiments, the polynucleotide comprises a nucleic acid sequence with 70% to 100% homology or identity to SEQ ID NO: 10. Each possibility represents a separate embodiment of the invention.

In some embodiments, the polynucleotide encoding transcobalamin 2 comprises or consists of a nucleic acid sequence set forth in SEQ ID NO: 10. In some embodiments, the polynucleotide further comprises a nucleic acid sequence encoding a signal or a leading peptide. In some embodiments, the signal peptide is directing the transcobalamin polypeptide for secretion from the cell.

In some embodiments, the polynucleotide comprises or consists of the nucleic acid sequence:

(SEQ ID NO: 11)
ATGAAGTTTTTCGCCATTGCCGCCCTATTTGCCGCCGCTGCCGTTGCCCA
GCCTAACGTCATCTCGAAGCGGCAGATCTGTGTGGTAAATGAAACGAACT
TCTGGCGTCTAGAACCTCTGATAAACACGATGCTGAATGCAAACTACACC
AAAGGGACCCTAGGTCTCAATGTCCTGTTGACCTTGAGACTTGTTGGGAT
TAAGAATCAAAGCCTAGAACAACAGCTGAGTCAACAAATCAAAGAAGACA
TAAACAGCGGAGAGATGGAGTTAACCTCGGGAGAGCTTGCCTTGGGTATT
CTGGGTTTGGGAGCATGTGAAAATCAGGATGAAGAGTTTATACGTGGTGC
TCGTCTGGTTAGCAAACTTGAAGCCAAATTCCAAGCAGAAATTCAGAACA
TGGAGGCACATGACGGCAACCCACTGACCAACTACTACCAGCTCAGCCTG
GCCCTGCTGGCCTTGTGTCTGTTCAACGGCAGCTATTCAGTCACCAGTGT
TACCTGTTACTTCACTCCTGAAAATAAGAACTATTATTTTGGGGACCAGT
TCTCAGTAGATACTGGAGCGATGGCTGTTCTTGCTTTAACATGTGTGCAG
AAGAATACACAAAAGAATATGGACATGAACGCAGAGGGTAAAATCAGTAA
TTACGTGGCGTCACTGGCAAACAAGATCCAGGCTGAGAGAAAACAGGGTC
TCTTTGGAAACATATTTAGCACGGGAGAAGCTATGCAGGCTCTCTTTGTC
TCATCGGACTACTACAAAAATGAAGCAAATTGCAGAGAAACTCTACGTGC
AGTATTTGACAACATTTCTCGGGGAGCATTTTACTTGCCGATTACTGCAG
CCCAGATCTTACCTGCCTTGATGGGAAAGACCTATCTGGATGTCACCAAC
CCTTCTTGTGGCCTCAATCCAGTCAAATTCAACACCTCCACTGAAAAGCC
TGGTACTGTGACACCTACAACCGCACCCTTGAATATCTTAGTCAAATACT
CTGTGCGAATCAACAAGACAAGTCACACTGAAGTCACTGTGAGAAAGGGT
TCTGTCTTCCTCGACGTGATGAAGGCTGCTCAGGAGAAAAATGAGACCCT
ATTTCGCTTCACAGTGGAGGAGACCTCGTGGGGCCCCTACATCACCTCTG
TTCAGGGCATAAAGGCCAGCAATAATGACAGAACCTACTGGAAGCTTCTG
AGCAACGGCCAGCCACTGAGCCAAGGAGCTGGGAGTCATGTTGTCCAAAA
TGGAGACAATTTGGAGGTTCGTTGGAGCAAATACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 11, or any value and range therebetween. Each possibility represents a separate embodiment of the invention. In some embodiments, the polynucleotide comprises a nucleic acid sequence with 70% to 100% homology or identity to SEQ ID NO: 11. Each possibility represents a separate embodiment of the invention.

In some embodiments, the polynucleotide encoding transcobalamin 1 comprises or consists of a nucleic acid sequence set forth in SEQ ID NO: 11.

In some embodiments, the polynucleotide comprises or consists of the nucleic acid sequence:

(SEQ ID NO: 12)
ATGAAGTTCTTCGCCATTGCCGCCCTATTTGCCGCCGCCGCCGTCGCCCA
GCCCAACGTCATCTCCAAGCGCCAGATCTGCGTCGTCAACGAGACCAACT
TCTGGCGCCTCGAGCCCCTCATCAACACCATGCTCAACGCCAACTACACC
AAGGGTACCCTCGGTCTCAACGTCCTCCTCACCCTCCGCCTCGTCGGTAT
CAAGAACCAGTCCCTCGAGCAGCAGCTCTCCCAGCAGATCAAGGAGGACA
TCAACTCCGGTGAGATGGAGCTCACCTCCGGTGAGCTCGCCCTCGGTATC
CTCGGTCTCGGTGCCTGCGAGAACCAGGACGAGGAGTTCATCCGCGGTGC
CCGCCTCGTCTCCAAGCTCGAGGCCAAGTTCCAAGCCGAGATCCAAAACA
TGGAGGCCCACGACGGTAACCCCCTCACCAACTACTACCAGCTCTCCCTC
GCCCTCCTCGCCCTCTGCCTCTTCAACGGTTCCTACTCCGTCACCTCCGT
CACCTGCTACTTCACCCCCGAGAACAAGAACTACTACTTCGGTGACCAGT
TCTCCGTCGACACCGGTGCCATGGCCGTCCTCGCCCTCACCTGCGTCCAA
AAGAACACCCAGAAGAACATGGACATGAACGCCGAGGGTAAAATCTCCAA
CTACGTCGCCTCCCTCGCCAACAAGATCCAAGCCGAGCGCAAGCAGGGTC
TCTTCGGTAACATCTTCTCCACCGGTGAGGCCATGCAAGCCCTCTTCGTC
TCCTCCGACTACTACAAGAACGAGGCCAACTGCCGCGAGACCCTCCGCGC
CGTCTTCGACAACATCTCCCGCGGTGCCTTCTACCTCCCCATCACCGCCG
CCCAGATCCTCCCCGCCCTCATGGGTAAAACCTACCTCGACGTCACCAAC
CCCTCCTGCGGTCTCAACCCCGTCAAGTTCAACACCTCCACCGAGAAGCC
CGGTACCGTCACCCCCACCACCGCCCCCCTCAACATCCTCGTCAAGTACT
CCGTCCGCATCAACAAGACCTCCCACACCGAGGTCACCGTCCGCAAGGGT
TCCGTCTTCCTCGACGTCATGAAGGCCGCCCAGGAGAAGAACGAGACCCT
CTTCCGCTTCACCGTCGAGGAGACCTCCTGGGGTCCCTACATCACCTCCG
TCCAAGGTATCAAGGCCTCCAACAACGACCGCACCTACTGGAAGCTCCTC
TCCAACGGTCAGCCCCTCTCCCAGGGTGCCGGTTCCCACGTCGTCCAAAA
CGGTGACAACCTCGAGGTCCGCTGGTCCAAGTACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 12, or any value and range therebetween. Each possibility represents a separate embodiment of the invention. In some embodiments, the polynucleotide comprises a nucleic acid sequence with 70% to 100% homology or identity to SEQ ID NO: 12. Each possibility represents a separate embodiment of the invention.

In some embodiments, the polynucleotide encoding transcobalamin 1 comprises or consists of a nucleic acid sequence set forth in SEQ ID NO: 12.

In some embodiments, the polynucleotide comprises or consists of the nucleic acid sequence:

(SEQ ID NO: 13)
ATGAAGTTTTTTGCCATTGCTGCCTTATTTGCCGCGGCTGCCGTCGCCCA
GCCTAACGTCATCTCCAAGAGACAGATCTGTGTCGTCAACGAGACCAACT
TCTGGCGTCTGGAGCCGTTGATCAACACCATGCTCAACGCCAACTACACC
AAGGGTACCCTCGGTCTCAACGTCCTCCTCACCCTCCGCCTCGTCGGTAT
CAAGAACCAGTCCCTCGAGCAGCAGCTCTCCCAGCAGATCAAGGAGGACA
TCAACTCCGGTGAGATGGAGCTCACCTCCGGTGAGCTCGCCCTCGGTATC
CTCGGTCTCGGTGCCTGCGAGAACCAGGACGAGGAGTTCATCCGCGGTGC
CCGCCTCGTCTCCAAGCTCGAGGCCAAGTTCCAGGCCGAGATCCAGAACA
TGGAGGCCCACGACGGTAACCCCCTCACCAACTACTACCAGCTCTCCCTC
GCCCTCCTCGCCCTCTGCCTCTTCAACGGTTCCTACTCCGTCACCTCCGT
CACCTGCTACTTCACCCCCGAGAACAAGAACTACTACTTCGGTGACCAGT
TCTCCGTCGACACCGGTGCCATGGCCGTCCTCGCCCTCACCTGCGTCCAG
AAGAACACCCAGAAGAACATGGACATGAACGCCGAGGGTAAGATCTCCAA
CTACGTCGCCTCCCTCGCCAACAAGATCCAGGCCGAGCGCAAGCAGGGTC
TCTTCGGTAACATCTTCTCCACCGGTGAGGCCATGCAGGCCCTCTTCGTC
TCCTCCGACTACTACAAGAACGAGGCCAACTGCCGCGAGACCCTCCGCGC
CGTCTTCGACAACATCTCCCGCGGTGCCTTCTACCTCCCCATCACCGCCG
CCCAGATCCTCCCCGCCCTCATGGGTAAGACCTACCTCGACGTCACCAAC
CCCTCCTGCGGTCTCAACCCCGTCAAGTTCAACACCTCCACCGAGAAGCC
CGGTACCGTCACCCCCACCACCGCCCCCCTCAACATCCTCGTCAAGTACT
CCGTCCGCATCAACAAGACCTCCCACACCGAGGTCACCGTCCGCAAGGGT
TCCGTCTTCCTCGACGTCATGAAGGCCGCCCAGGAGAAGAACGAGACCCT
CTTCCGCTTCACCGTCGAGGAGACCTCCTGGGGTCCCTACATCACCTCCG
TCCAGGGTATCAAGGCCTCCAACAACGACCGCACCTACTGGAAGCTCCTC
TCCAACGGTCAGCCCCTCTCCCAGGGTGCCGGTTCCCACGTCGTCCAGAA
CGGTGACAACCTCGAGGTCCGCTGGTCCAAGTACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 13, or any value and range therebetween. Each possibility represents a separate embodiment of the invention. In some embodiments, the polynucleotide comprises a nucleic acid sequence with 70% to 100% homology or identity to SEQ ID NO: 13. Each possibility represents a separate embodiment of the invention.

In some embodiments, the polynucleotide encoding transcobalamin 1 comprises or consists of a nucleic acid sequence set forth in SEQ ID NO: 13.

In some embodiments, the polynucleotide comprises or consists of the nucleic acid sequence:

(SEQ ID NO: 14)
ATGAAGTTTTTTGCCATTGCTGCCTTATTTGCCGCGGCTGCCGTCGCCCA
GCCTAACGTCATCTCCAAGAGACAGATCTGTGTCGTCAACGAGACCAACT
TCTGGCGTCTGGAGCCGTTGATTAACACCATGTTGAACGCCAACTACACC
AAAGGCACGCTCGGTCTCAATGTTCTGCTCACCCTCCGTCTGGTCGGTAT
CAAGAACCAAAGCCTAGAACAACAGCTCTCCCAGCAAATCAAAGAGGACA
TCAACAGCGGCGAGATGGAACTAACTTCCGGCGAGCTTGCGCTCGGTATC
CTCGGTCTCGGTGCTTGCGAGAATCAAGATGAAGAATTCATTCGTGGCGC
TAGATTGGTTTCGAAGTTGGAGGCCAAGTTCCAGGCGGAGATCCAGAACA
TGGAGGCACATGACGGTAACCCTCTCACCAACTACTACCAGCTCTCCCTC
GCACTACTTGCATTGTGCCTCTTCAACGGCAGCTATAGCGTTACTTCCGT
CACCTGTTATTTCACCCCCGAGAACAAGAACTACTATTTCGGTGACCAGT
TCTCTGTCGACACTGGTGCCATGGCCGTTCTCGCCCTCACCTGCGTACAG
AAGAACACACAGAAGAACATGGACATGAACGCCGAGGGCAAGATCTCGAA
CTACGTTGCTTCCCTCGCCAACAAGATCCAAGCCGAGAGGAAGCAAGGCC
TATTCGGCAACATCTTCTCCACCGGCGAGGCCATGCAGGCGCTGTTTGTC
TCTTCGGACTACTATAAGAACGAGGCAAATTGCCGTGAGACTTTGCGCGC
TGTGTTCGACAACATCTCCCGCGGTGCCTTCTACCTGCCTATCACCGCCG
CCCAGATTCTGCCCGCTCTCATGGGAAAGACCTACCTCGACGTCACCAAC
CCCTCCTGCGGTCTCAACCCCGTCAAGTTCAATACCAGCACCGAGAAGCC
TGGTACCGTCACGCCAACTACTGCTCCTCTCAACATCCTTGTCAAGTACT
CGGTCCGCATTAACAAGACCAGCCACACCGAGGTCACCGTCCGCAAGGGC
TCCGTCTTCTTGGATGTTATGAAGGCCGCCCAGGAGAAAAACGAGACCCT
CTTCCGCTTCACCGTGGAGGAGACCTCCTGGGGTCCCTACATCACATCCG
TTCAGGGTATCAAAGCTTCCAATAATGACCGCACCTACTGGAAGCTCCTC
TCCAACGGCCAGCCACTGTCGCAAGGTGCTGGTTCTCATGTGGTTCAGAA
CGGCGACAACCTGGAGGTCCGCTGGAGCAAGTACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 14, or any value and range therebetween. Each possibility represents a separate embodiment of the invention. In some embodiments, the polynucleotide comprises a nucleic acid sequence with 70% to 100% homology or identity to SEQ ID NO: 14. Each possibility represents a separate embodiment of the invention.

In some embodiments, the polynucleotide encoding transcobalamin 1 comprises or consists of a nucleic acid sequence set forth in SEQ ID NO: 14.

In some embodiments, the polynucleotide comprises or consists of the nucleic acid sequence:

(SEQ ID NO: 15)
ATGAAGTTCTTTGCCATTGCTGCCTTATTTGCCGCGGCTGCCGTCGCCCA
GCCTAACGTCATCTCCAAGAGACAGATCTGTGTCGTCAACGAGACCAACT
TCTGGCGTCTGGAGCCGTTGATTAACACCATGTTGAACGCCAACTACACC
AAAGGCACGCTCGGTCTCAATGTTCTGCTCACCCTCCGTCTGGTCGGTAT
CAAGAACCAAAGCCTAGAACAACAGCTCTCCCAGCAAATCAAAGAGGACA
TCAACAGCGGCGAGATGGAACTAACTTCCGGCGAGCTTGCGCTCGGTATC
CTCGGTCTCGGTGCTTGCGAGAATCAAGATGAAGAATTCATTCGTGGCGC
TAGATTGGTTTCGAAGTTGGAGGCCAAGTTCCAGGCGGAGATCCAGAACA
TGGAGGCACATGACGGTAACCCTCTCACCAACTACTACCAGCTCTCCCTC
GCACTACTTGCATTGTGCCTCTTCAACGGCAGCTATAGCGTTACTTCCGT
CACCTGTTATTTCACCCCCGAGAACAAGAACTACTATTTCGGTGACCAGT
TCTCTGTCGACACTGGTGCCATGGCCGTTCTCGCCCTCACCTGCGTACAG
AAGAACACACAGAAGAACATGGACATGAACGCCGAGGGCAAGATCTCGAA
CTACGTTGCTTCCCTCGCCAACAAGATCCAAGCCGAGAGGAAGCAAGGCC
TATTCGGCAACATCTTCTCCACCGGCGAGGCCATGCAGGCGCTGTTTGTC
TCTTCGGACTACTATAAGAACGAGGCAAATTGCCGTGAGACTTTGCGCGC
TGTGTTCGACAACATCTCCCGCGGTGCCTTCTACCTGCCTATCACCGCCG
CCCAGATTCTGCCCGCTCTCATGGGAAAGACCTACCTCGACGTCACCAAC
CCCTCCTGCGGTCTCAACCCCGTCAAGTTCAATACCAGCACCGAGAAGCC
TGGTACCGTCACGCCAACTACTGCTCCTCTCAACATCCTTGTCAAGTACT
CGGTCCGCATTAACAAGACCAGCCACACCGAGGTCACCGTCCGCAAGGGC
TCCGTCTTCTTGGATGTTATGAAGGCCGCCCAGGAGAAAAACGAGACCCT
CTTCCGCTTCACCGTGGAGGAGACCTCCTGGGGTCCCTACATCACATCCG
TTCAGGGTATCAAAGCTTCCAATAATGACCGCACCTACTGGAAGCTCCTC
TCCAACGGCCAGCCACTGTCGCAAGGTGCTGGTTCTCATGTGGTTCAGAA
CGGCGACAACCTGGAGGTCCGCTGGAGCAAGTACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 15, or any value and range therebetween. Each possibility represents a separate embodiment of the invention. In some embodiments, the polynucleotide comprises a nucleic acid sequence with 70% to 100% homology or identity to SEQ ID NO: 15. Each possibility represents a separate embodiment of the invention.

In some embodiments, the polynucleotide encoding transcobalamin 1 comprises or consists of a nucleic acid sequence set forth in SEQ ID NO: 15.

In some embodiments, the polynucleotide comprises or consists of the nucleic acid sequence:

(SEQ ID NO: 16)
ATGAAGTTCTTTGCCATTGCTGCCTTATTTGCCGCGGCTGCCGTCGCCCA
GCCTAACGTCATCTCCAAGAGACAGATCTGTGTCGTCAACGAGACCAACT
TCTGGCGTCTGGAGCCGTTGATCAACACCATGCTCAACGCCAACTACACC
AAGGGTACCCTCGGTCTCAACGTCCTCCTCACCCTCCGCCTCGTCGGTAT
CAAGAACCAGTCCCTCGAGCAGCAGCTCTCCCAGCAGATCAAGGAGGACA
TCAACTCCGGTGAGATGGAGCTCACCTCCGGTGAGCTCGCCCTCGGTATC
CTCGGTCTCGGTGCCTGCGAGAACCAGGACGAGGAGTTCATCCGCGGTGC
CCGCCTCGTCTCCAAGCTCGAGGCCAAGTTCCAGGCCGAGATCCAGAACA
TGGAGGCCCACGACGGTAACCCCCTCACCAACTACTACCAGCTCTCCCTC
GCCCTCCTCGCCCTCTGCCTCTTCAACGGTTCCTACTCCGTCACCTCCGT
CACCTGCTACTTCACCCCCGAGAACAAGAACTACTACTTCGGTGACCAGT
TCTCCGTCGACACCGGTGCCATGGCCGTCCTCGCCCTCACCTGCGTCCAG
AAGAACACCCAGAAGAACATGGACATGAACGCCGAGGGTAAGATCTCCAA
CTACGTCGCCTCCCTCGCCAACAAGATCCAGGCCGAGCGCAAGCAGGGTC
TCTTCGGTAACATCTTCTCCACCGGTGAGGCCATGCAGGCCCTCTTCGTC
TCCTCCGACTACTACAAGAACGAGGCCAACTGCCGCGAGACCCTCCGCGC
CGTCTTCGACAACATCTCCCGCGGTGCCTTCTACCTCCCCATCACCGCCG
CCCAGATCCTCCCCGCCCTCATGGGTAAGACCTACCTCGACGTCACCAAC
CCCTCCTGCGGTCTCAACCCCGTCAAGTTCAACACCTCCACCGAGAAGCC
CGGTACCGTCACCCCCACCACCGCCCCCCTCAACATCCTCGTCAAGTACT
CCGTCCGCATCAACAAGACCTCCCACACCGAGGTCACCGTCCGCAAGGGT
TCCGTCTTCCTCGACGTCATGAAGGCCGCCCAGGAGAAGAACGAGACCCT
CTTCCGCTTCACCGTCGAGGAGACCTCCTGGGGTCCCTACATCACCTCCG
TCCAGGGTATCAAGGCCTCCAACAACGACCGCACCTACTGGAAGCTCCTC
TCCAACGGTCAGCCCCTCTCCCAGGGTGCCGGTTCCCACGTCGTCCAGAA
CGGTGACAACCTCGAGGTCCGCTGGTCCAAGTACTAA.

In some embodiments, the polynucleotide comprises a nucleic acid sequence with at least 70%, at least 80%, at least 90%, at least 95%, at least 97%, at least 99%, or 100% homology or identity to SEQ ID NO: 16, or any value and range therebetween. Each possibility represents a separate embodiment of the invention. In some embodiments, the polynucleotide comprises a nucleic acid sequence with 70% to 100% homology or identity to SEQ ID NO: 16. Each possibility represents a separate embodiment of the invention.

In some embodiments, the polynucleotide encoding transcobalamin 1 comprises or consists of a nucleic acid sequence set forth in SEQ ID NO: 16.

In some embodiments, the polynucleotide comprises or consists of the nucleic acid sequence:

(SEQ ID NO: 17)
ATGAAGTTTTTCGCCATTGCCGCCCTATTTGCCGCCGCTGCCGTTGCCCA
GCCTAACGTCATCTCGAAGCGGAACATCTGCGAGATAACCGAGGTGGACA
GCACGCTGGTGGAGAGGCTGGGCCAGCGCCTCTTGCCCTGGATGGACCGG
CTCTCCCAGGAGCAGCTGAACCCCAGTATCTACGTGGGCCTGCGCCTCTC
GAGCCTGCAGGCTGGGGCCAAGGAGGCCCACTACCTGCACAGCCTCAAGC
TCAGCTACCAGCAGAGCCTCCTGAGGCCTGCCTCCAACAAGGATGACAAT
GACTCCGAGGCCAAGCCCTCTATGGGCCAGCTGGCCCTCTACCTGCTGGC
TCTCCGGGCCAACTGCGAGTTCATCGGAGGCCGCAAGGGGGACAGGCTGG
TCTCCCAGCTGAAGCGGTTCCTGGAGGACGAGAAGAGGGCCATCGGGCAC
AACCACCAGGGTCACCCCCGCACCAGCTACTACCAGTACAGCCTGGGCAT
CCTGGCCCTGTGTGTCCACCAGAAGCGAGTCCACGACAGTGTGGTGGGCA
AGCTCCTGTACGCCGTGGAACACAAGCCGCATCTCCTGCAGGACCACGTC
TCTGTGGACACCATGGCCATGGCAGGCATGGCCTTCTCCTGTCTGGAGCT
GTCCAACCTCAACCCCAAGCAGAGAAACCGGATCAACCTGGCCCTCAAGA
GAGTGCAAGAGAAGATCCTGAAGGCCCAGACCCCAGAGGGCTACTTCGGG
AATGTCTACAGCACCCCTCTGGCTTTGCAGTTGCTGATGGGCTCCCTCAG
GCCCTCGGTGGAGCTGGGCACAGCCTGCCTTAAAGCCAAGGCTGCTCTGC
AGGCCAGCCTACAGCACAAGACCTTCCAGAACCCTCTCATGATCTCTCAG
CTGCTGCCTGTCCTGAACCAGAAGAGCTATGTGGATCTCATCTCCCCAGA
CTGCCAGGCTCCAAGAGCCCTGTTGGAACCGGCTCTGGAGACCCCGCCAC
AGGCCAAAGTCCCGAAGTTCATTGACGTCTTGCTGAAGGTCTCCGGCATC
TCCCCTTCATACAGACACTCTGTCTCTGTCCCTGCTGGCTCCTCCCTGGA
AGACATCCTGAAGAACGCCCAGGAGCATGGAAGATTCAGGTTTAGAACAC
AGGCCTCCCTGTCTGGCCCCTTCCTGACCTCCGTGCTGGGGAGAAAGGCT
GGGGAACGTGAGTTCTGGCAGGTCCTCCGAGATCCTGACACCCCCTTGCA
GCAAGGTATTGCTGACTACAGACCCAAGGATGGAGAGACCATCGAGCTGA
GGCTGGTTGGCTGGTAG.

In some embodiments, the polynucleotide comprises a nucleic acid sequence with at least 70%, at least 80%, at least 90%, at least 95%, at least 97%, at least 99%, or 100% homology or identity to SEQ ID NO: 17, or any value and range therebetween. Each possibility represents a separate embodiment of the invention. In some embodiments, the polynucleotide comprises a nucleic acid sequence with 70% to 100% homology or identity to SEQ ID NO: 17. Each possibility represents a separate embodiment of the invention.

In some embodiments, the polynucleotide encoding transcobalamin 2 comprises or consists of a nucleic acid sequence set forth in SEQ ID NO: 17.

In some embodiments, the polynucleotide comprises or consists of the nucleic acid sequence:

(SEQ ID NO: 18)
ATGAAGTTCTTCGCCATTGCCGCCCTATTTGCCGCCGCCGCCGTCGCCCA
GCCCAACGTCATCTCCAAGCGCAACATCTGCGAGATCACCGAGGTCGACT
CCACCCTCGTCGAGCGCCTCGGTCAGCGCCTCCTCCCCTGGATGGACCGC
CTCTCCCAGGAGCAGCTCAACCCCTCCATCTACGTCGGTCTCCGCCTCTC
CTCCCTCCAAGCCGGTGCCAAGGAGGCCCACTACCTCCACTCCCTCAAGC
TCTCCTACCAGCAGTCCCTCCTCCGCCCCGCCTCCAACAAGGACGACAAC
GACTCCGAGGCCAAGCCCTCCATGGGTCAGCTCGCCCTCTACCTCCTCGC
CCTCCGCGCCAACTGCGAGTTCATCGGTGGTCGCAAGGGTGACCGCCTCG
TCTCCCAGCTCAAGCGCTTCCTCGAGGACGAGAAGCGCGCCATCGGTCAC
AACCACCAGGGTCACCCCCGCACCTCCTACTACCAGTACTCCCTCGGTAT
CCTCGCCCTCTGCGTCCACCAGAAGCGCGTCCACGACTCCGTCGTCGGTA
AACTCCTCTACGCCGTCGAGCACAAGCCCCACCTCCTCCAAGACCACGTC
TCCGTCGACACCATGGCCATGGCCGGTATGGCCTTCTCCTGCCTCGAGCT
CTCCAACCTCAACCCCAAGCAGCGCAACCGCATCAACCTCGCCCTCAAGC
GCGTCCAAGAGAAGATCCTCAAGGCCCAGACCCCCGAGGGTTACTTCGGT
AACGTCTACTCCACCCCCCTCGCCCTCCAACTCCTCATGGGTTCCCTCCG
CCCCTCCGTCGAGCTCGGTACCGCCTGCCTCAAGGCCAAGGCCGCCCTCC
AAGCCTCCCTCCAACACAAGACCTTCCAAAACCCCCTCATGATCTCCCAG
CTCCTCCCCGTCCTCAACCAGAAGTCCTACGTCGACCTCATCTCCCCCGA
CTGCCAGGCCCCCCGCGCCCTCCTCGAGCCCGCCCTCGAGACCCCCCCCC
AGGCCAAGGTCCCCAAGTTCATCGACGTCCTCCTCAAGGTCTCCGGTATC
TCCCCCTCCTACCGCCACTCCGTCTCCGTCCCCGCCGGTTCCTCCCTCGA
GGACATCCTCAAGAACGCCCAGGAGCACGGTCGCTTCCGCTTCCGCACCC
AGGCCTCCCTCTCCGGTCCCTTCCTCACCTCCGTCCTCGGTCGCAAGGCC
GGTGAGCGCGAGTTCTGGCAGGTCCTCCGCGACCCCGACACCCCCCTCCA
GCAGGGTATCGCCGACTACCGCCCCAAGGACGGTGAGACCATCGAGCTCC
GCCTCGTCGGTTGGTAA.

In some embodiments, the polynucleotide comprises a nucleic acid sequence with at least 70%, at least 80%, at least 90%, at least 95%, at least 97%, at least 99%, or 100% homology or identity to SEQ ID NO: 18, or any value and range therebetween. Each possibility represents a separate embodiment of the invention. In some embodiments, the polynucleotide comprises a nucleic acid sequence with 70% to 100% homology or identity to SEQ ID NO: 18. Each possibility represents a separate embodiment of the invention.

In some embodiments, the polynucleotide encoding transcobalamin 2 comprises or consists of a nucleic acid sequence set forth in SEQ ID NO: 18.

In some embodiments, the polynucleotide comprises or consists of the nucleic acid sequence:

(SEQ ID NO: 19)
ATGAAGTTTTTTGCCATTGCTGCCTTATTTGCCGCGGCTGCCGTCGCCCA
GCCTAACGTCATCTCCAAGCGTAACATCTGTGAGATCACTGAAGTGGACT
CTACTCTCGTTGAGCGCTTGGGTCAGCGCCTCCTCCCCTGGATGGACCGC
CTCTCCCAGGAGCAGCTCAACCCCTCCATCTACGTCGGTCTCCGCCTCTC
CTCCCTCCAGGCCGGTGCCAAGGAGGCCCACTACCTCCACTCCCTCAAGC
TCTCCTACCAGCAGTCCCTCCTCCGCCCCGCCTCCAACAAGGACGACAAC
GACTCCGAGGCCAAGCCCTCCATGGGTCAGCTCGCCCTCTACCTCCTCGC
CCTCCGCGCCAACTGCGAGTTCATCGGTGGTCGCAAGGGTGACCGCCTCG
TCTCCCAGCTCAAGCGCTTCCTCGAGGACGAGAAGCGCGCCATCGGTCAC
AACCACCAGGGTCACCCCCGCACCTCCTACTACCAGTACTCCCTCGGTAT
CCTCGCCCTCTGCGTCCACCAGAAGCGCGTCCACGACTCCGTCGTCGGTA
AGCTCCTCTACGCCGTCGAGCACAAGCCCCACCTCCTCCAGGACCACGTC
TCCGTCGACACCATGGCCATGGCCGGTATGGCCTTCTCCTGCCTCGAGCT
CTCCAACCTCAACCCCAAGCAGCGCAACCGCATCAACCTCGCCCTCAAGC
GCGTCCAGGAGAAGATCCTCAAGGCCCAGACCCCCGAGGGTTACTTCGGT
AACGTCTACTCCACCCCCCTCGCCCTCCAGCTCCTCATGGGTTCCCTCCG
CCCCTCCGTCGAGCTCGGTACCGCCTGCCTCAAGGCCAAGGCCGCCCTCC
AGGCCTCCCTCCAGCACAAGACCTTCCAGAACCCCCTCATGATCTCCCAG
CTCCTCCCCGTCCTCAACCAGAAGTCCTACGTCGACCTCATCTCCCCCGA
CTGCCAGGCCCCCCGCGCCCTCCTCGAGCCCGCCCTCGAGACCCCCCCCC
AGGCCAAGGTCCCCAAGTTCATCGACGTCCTCCTCAAGGTCTCCGGTATC
TCCCCCTCCTACCGCCACTCCGTCTCCGTCCCCGCCGGTTCCTCCCTCGA
GGACATCCTCAAGAACGCCCAGGAGCACGGTCGCTTCCGCTTCCGCACCC
AGGCCTCCCTCTCCGGTCCCTTCCTCACCTCCGTCCTCGGTCGCAAGGCC
GGTGAGCGCGAGTTCTGGCAGGTCCTCCGCGACCCCGACACCCCCCTCCA
GCAGGGTATCGCCGACTACCGCCCCAAGGACGGTGAGACCATCGAGCTCC
GCCTCGTCGGTTGGTAA.

In some embodiments, the polynucleotide comprises a nucleic acid sequence with at least 70%, at least 80%, at least 90%, at least 95%, at least 97%, at least 99%, or 100% homology or identity to SEQ ID NO: 19, or any value and range therebetween. Each possibility represents a separate embodiment of the invention. In some embodiments, the polynucleotide comprises a nucleic acid sequence with 70% to 100% homology or identity to SEQ ID NO: 19. Each possibility represents a separate embodiment of the invention.

In some embodiments, the polynucleotide encoding transcobalamin 2 comprises or consists of a nucleic acid sequence set forth in SEQ ID NO: 19.

In some embodiments, the polynucleotide comprises or consists of the nucleic acid sequence:

(SEQ ID NO: 20)
ATGAAGTTTTTTGCCATTGCTGCCTTATTTGCCGCGGCTGCCGTCGCCCA
GCCTAACGTCATCTCCAAGCGTAACATCTGTGAGATCACTGAAGTGGACT
CTACTCTCGTTGAGCGCTTGGGTCAACGCCTACTTCCCTGGATGGACAGA
CTCTCTCAGGAGCAGCTGAACCCGAGCATCTATGTCGGCCTCCGTCTGTC
ATCCCTCCAGGCTGGTGCTAAGGAAGCGCACTACCTCCACTCTCTGAAAC
TGTCATACCAGCAGTCGCTCCTCCGCCCCGCTTCCAACAAGGACGATAAT
GACAGCGAGGCTAAACCATCTATGGGTCAGCTAGCCCTCTATCTGCTTGC
CCTGCGGGCAAACTGCGAGTTCATAGGCGGAAGAAAGGGTGACCGTCTCG
TCTCCCAGTTGAAGAGATTTCTGGAAGACGAAAAGAGGGCTATCGGCCAC
AACCACCAGGGCCACCCCAGAACCTCCTACTACCAGTACTCGCTGGGTAT
CCTCGCCCTCTGCGTTCATCAGAAGCGTGTCCACGATTCCGTTGTCGGTA
AGCTCCTCTACGCAGTGGAACACAAGCCCCATTTGCTTCAAGACCACGTG
AGTGTCGACACCATGGCCATGGCGGGTATGGCATTCAGCTGCTTGGAGCT
CTCCAACCTAAACCCCAAACAGCGTAACCGCATCAACCTAGCGTTGAAAC
GAGTGCAAGAGAAGATCCTTAAGGCCCAAACCCCCGAAGGCTACTTCGGT
AACGTCTACTCCACACCCCTCGCCCTCCAGCTCCTCATGGGGAGCCTTAG
ACCATCCGTCGAATTAGGAACCGCTTGCTTGAAAGCAAAGGCAGCCCTGC
AAGCATCTCTCCAGCACAAGACTTTCCAGAATCCACTCATGATTTCTCAG
CTGCTTCCCGTTCTCAACCAGAAGTCGTATGTTGACCTCATTAGCCCGGA
TTGCCAGGCACCACGAGCTTTACTTGAGCCTGCACTGGAGACCCCGCCCC
AGGCCAAGGTCCCTAAGTTCATCGATGTGTTGCTCAAGGTCAGTGGTATC
AGCCCCTCCTATCGTCACTCCGTGAGCGTGCCCGCTGGAAGCTCTTTGGA
AGATATCCTTAAGAATGCCCAGGAGCACGGTAGGTTCAGGTTTCGTACCC
AAGCCTCTCTTTCCGGCCCCTTCCTCACCAGCGTACTTGGCCGTAAGGCC
GGCGAGCGTGAGTTCTGGCAGGTGCTGCGTGATCCCGACACCCCACTGCA
ACAGGGCATTGCAGACTACCGACCGAAGGATGGAGAGACTATCGAGCTCC
GCCTTGTCGGTTGGTAA.

In some embodiments, the polynucleotide comprises a nucleic acid sequence with at least 70%, at least 80%, at least 90%, at least 95%, at least 97%, at least 99%, or 100% homology or identity to SEQ ID NO: 20, or any value and range therebetween. Each possibility represents a separate embodiment of the invention. In some embodiments, the polynucleotide comprises a nucleic acid sequence with 70% to 100% homology or identity to SEQ ID NO: 20. Each possibility represents a separate embodiment of the invention.

In some embodiments, the polynucleotide encoding transcobalamin 2 comprises or consists of a nucleic acid sequence set forth in SEQ ID NO: 20.

In some embodiments, the polynucleotide comprises or consists of the nucleic acid sequence:

(SEQ ID NO: 21)
ATGAAGTTCTTTGCCATTGCTGCCTTATTTGCCGCGGCTGCCGTCGCCCA
GCCTAACGTCATCTCCAAGCGTAACATCTGTGAGATCACTGAAGTGGACT
CTACTCTCGTTGAGCGCTTGGGTCAACGCCTACTTCCCTGGATGGACAGA
CTCTCTCAGGAGCAGCTGAACCCGAGCATCTATGTCGGCCTCCGTCTGTC
ATCCCTCCAGGCTGGTGCTAAGGAAGCGCACTACCTCCACTCTCTGAAAC
TGTCATACCAGCAGTCGCTCCTCCGCCCCGCTTCCAACAAGGACGATAAT
GACAGCGAGGCTAAACCATCTATGGGTCAGCTAGCCCTCTATCTGCTTGC
CCTGCGGGCAAACTGCGAGTTCATAGGCGGAAGAAAGGGTGACCGTCTCG
TCTCCCAGTTGAAGAGATTTCTGGAAGACGAAAAGAGGGCTATCGGCCAC
AACCACCAGGGCCACCCCAGAACCTCCTACTACCAGTACTCGCTGGGTAT
CCTCGCCCTCTGCGTTCATCAGAAGCGTGTCCACGATTCCGTTGTCGGTA
AGCTCCTCTACGCAGTGGAACACAAGCCCCATTTGCTTCAAGACCACGTG
AGTGTCGACACCATGGCCATGGCGGGTATGGCATTCAGCTGCTTGGAGCT
CTCCAACCTAAACCCCAAACAGCGTAACCGCATCAACCTAGCGTTGAAAC
GAGTGCAAGAGAAGATCCTTAAGGCCCAAACCCCCGAAGGCTACTTCGGT
AACGTCTACTCCACACCCCTCGCCCTCCAGCTCCTCATGGGGAGCCTTAG
ACCATCCGTCGAATTAGGAACCGCTTGCTTGAAAGCAAAGGCAGCCCTGC
AAGCATCTCTCCAGCACAAGACTTTCCAGAATCCACTCATGATTTCTCAG
CTGCTTCCCGTTCTCAACCAGAAGTCGTATGTTGACCTCATTAGCCCGGA
TTGCCAGGCACCACGAGCTTTACTTGAGCCTGCACTGGAGACCCCGCCCC
AGGCCAAGGTCCCTAAGTTCATCGATGTGTTGCTCAAGGTCAGTGGTATC
AGCCCCTCCTATCGTCACTCCGTGAGCGTGCCCGCTGGAAGCTCTTTGGA
AGATATCCTTAAGAATGCCCAGGAGCACGGTAGGTTCAGGTTTCGTACCC
AAGCCTCTCTTTCCGGCCCCTTCCTCACCAGCGTACTTGGCCGTAAGGCC
GGCGAGCGTGAGTTCTGGCAGGTGCTGCGTGATCCCGACACCCCACTGCA
ACAGGGCATTGCAGACTACCGACCGAAGGATGGAGAGACTATCGAGCTCC
GCCTTGTCGGTTGGTAA.

In some embodiments, the polynucleotide comprises a nucleic acid sequence with at least 70%, at least 80%, at least 90%, at least 95%, at least 97%, at least 99%, or 100% homology or identity to SEQ ID NO: 21, or any value and range therebetween. Each possibility represents a separate embodiment of the invention. In some embodiments, the polynucleotide comprises a nucleic acid sequence with 70% to 100% homology or identity to SEQ ID NO: 21. Each possibility represents a separate embodiment of the invention.

In some embodiments, the polynucleotide encoding transcobalamin 2 comprises or consists of a nucleic acid sequence set forth in SEQ ID NO: 21.

In some embodiments, the polynucleotide comprises or consists of the nucleic acid sequence:

(SEQ ID NO: 22)
ATGAAGTTCTTTGCCATTGCTGCCTTATTTGCCGCGGCTGCCGTCGCCCA
GCCTAACGTCATCTCCAAGCGTAACATCTGTGAGATCACTGAAGTGGACT
CTACTCTCGTTGAGCGCTTGGGTCAGCGCCTCCTCCCCTGGATGGACCGC
CTCTCCCAGGAGCAGCTCAACCCCTCCATCTACGTCGGTCTCCGCCTCTC
CTCCCTCCAGGCCGGTGCCAAGGAGGCCCACTACCTCCACTCCCTCAAGC
TCTCCTACCAGCAGTCCCTCCTCCGCCCCGCCTCCAACAAGGACGACAAC
GACTCCGAGGCCAAGCCCTCCATGGGTCAGCTCGCCCTCTACCTCCTCGC
CCTCCGCGCCAACTGCGAGTTCATCGGTGGTCGCAAGGGTGACCGCCTCG
TCTCCCAGCTCAAGCGCTTCCTCGAGGACGAGAAGCGCGCCATCGGTCAC
AACCACCAGGGTCACCCCCGCACCTCCTACTACCAGTACTCCCTCGGTAT
CCTCGCCCTCTGCGTCCACCAGAAGCGCGTCCACGACTCCGTCGTCGGTA
AGCTCCTCTACGCCGTCGAGCACAAGCCCCACCTCCTCCAGGACCACGTC
TCCGTCGACACCATGGCCATGGCCGGTATGGCCTTCTCCTGCCTCGAGCT
CTCCAACCTCAACCCCAAGCAGCGCAACCGCATCAACCTCGCCCTCAAGC
GCGTCCAGGAGAAGATCCTCAAGGCCCAGACCCCCGAGGGTTACTTCGGT
AACGTCTACTCCACCCCCCTCGCCCTCCAGCTCCTCATGGGTTCCCTCCG
CCCCTCCGTCGAGCTCGGTACCGCCTGCCTCAAGGCCAAGGCCGCCCTCC
AGGCCTCCCTCCAGCACAAGACCTTCCAGAACCCCCTCATGATCTCCCAG
CTCCTCCCCGTCCTCAACCAGAAGTCCTACGTCGACCTCATCTCCCCCGA
CTGCCAGGCCCCCCGCGCCCTCCTCGAGCCCGCCCTCGAGACCCCCCCCC
AGGCCAAGGTCCCCAAGTTCATCGACGTCCTCCTCAAGGTCTCCGGTATC
TCCCCCTCCTACCGCCACTCCGTCTCCGTCCCCGCCGGTTCCTCCCTCGA
GGACATCCTCAAGAACGCCCAGGAGCACGGTCGCTTCCGCTTCCGCACCC
AGGCCTCCCTCTCCGGTCCCTTCCTCACCTCCGTCCTCGGTCGCAAGGCC
GGTGAGCGCGAGTTCTGGCAGGTCCTCCGCGACCCCGACACCCCCCTCCA
GCAGGGTATCGCCGACTACCGCCCCAAGGACGGTGAGACCATCGAGCTCC
GCCTCGTCGGTTGGTAA.

In some embodiments, the polynucleotide comprises a nucleic acid sequence with at least 70%, at least 80%, at least 90%, at least 95%, at least 97%, at least 99%, or 100% homology or identity to SEQ ID NO: 22, or any value and range therebetween. Each possibility represents a separate embodiment of the invention. In some embodiments, the polynucleotide comprises a nucleic acid sequence with 70% to 100% homology or identity to SEQ ID NO: 22. Each possibility represents a separate embodiment of the invention.

In some embodiments, the polynucleotide encoding transcobalamin 2 comprises or consists of a nucleic acid sequence set forth in SEQ ID NO: 22.

In some embodiments, the polynucleotide of the invention further comprises a nucleic acid sequence encoding a promotor. In some embodiments, the polynucleotide comprises or consists of the nucleic acid sequence:

(SEQ ID NO: 23)
GTTCAAAGACTAGGATGGATAAATGGGGTATATAAAGCACCCTGACTCCC
TTCCTCCAAGTTCTATCTAACCAGCCATCCTACACTCTACATATCCACAC
CAATCTACTAGTCAAC.

In some embodiments, the polynucleotide encoding a promotor comprises or consists of a nucleic acid sequence set forth in SEQ ID NO: 23.

In some embodiments, the nucleic acid sequence of the polynucleotide of the invention is codon optimized for expression in the cell.

In some embodiments, the nucleic acid sequence of the polynucleotide of the invention is codon optimized for expression in a fungal cell. In some embodiments, the fungal cell is Aspergillus orizae (A. orizae), Trichoderma reesei, or both. In some embodiments, codon optimization is for expression in A. orizae.

In some embodiments, the polynucleotide of the invention further comprises a nucleotide sequence encoding a signal peptide. In some embodiments, the polynucleotide comprises the nucleic acid sequence:

(SEQ ID NO: 24)
ATGAAGTTTTTCGCCATTGCCGCCCTATTTGCCGCCGCTGCCGTTGCC.

In some embodiments, the polynucleotide encoding a signal peptide comprises or consists of a nucleic acid sequence set forth in SEQ ID NO: 24.

In some embodiments, the polynucleotide comprises the nucleic acid sequence:

(SEQ ID NO: 25)
ATGATGGTCGCTTGGTGGAGTTTGTTTTTGTATGGGCTACAGGTAGCTGC
GCCGGCACTAGCA.

In some embodiments, the polynucleotide encoding a signal peptide comprises or consists of a nucleic acid sequence set forth in SEQ ID NO: 25.

In some embodiments, the polynucleotide comprises the nucleic acid sequence:

(SEQ ID NO: 26)
ATGCGTAATAATCTCTTATTTTCATTAAATGCCATCGCTGGAGCAGTAGC
GCACCCGTCTTTTCCGATCCATAAGCGA.

In some embodiments, the polynucleotide encoding a signal peptide comprises or consists of a nucleic acid sequence set forth in SEQ ID NO: 26.

In some embodiments, the polynucleotide comprises the nucleic acid sequence:

(SEQ ID NO: 27)
ATGCCTTGGGCCGGTTTAGCCACACTTTTTCTAGCAGTTGCGGGCGTACA
CGGG.

In some embodiments, the polynucleotide encoding a signal peptide comprises or consists of a nucleic acid sequence set forth in SEQ ID NO: 27.

In some embodiments, the polynucleotide comprises the nucleic acid sequence:

(SEQ ID NO: 28)
ATGCAGTTTTCCGTCGCCGCCGTCCTCGCTCTAGCTACCGCAGTCGCCGC
G.

In some embodiments, the polynucleotide encoding a signal peptide comprises or consists of a nucleic acid sequence set forth in SEQ ID NO: 28.

In some embodiments, the polynucleotide comprises the nucleic acid sequence:

(SEQ ID NO: 29)
ATGTCTCACGTCGAACACTTACGCCACACTGCACTGCAACTAAGGAGTCC
CCTATTTACCTGCCCCCATTTACCTGTA.

In some embodiments, the polynucleotide encoding a signal peptide comprises or consists of a nucleic acid sequence set forth in SEQ ID NO: 29.

In some embodiments, the polynucleotide comprises the nucleic acid sequence:

(SEQ ID NO: 30)
ATGACTCCTTCTTTATCTAAACTCGTTGCCCTTAGCTTATTCCTCGGGAC
TGCCTTAGGG.

In some embodiments, the polynucleotide encoding a signal peptide comprises or consists of a nucleic acid sequence set forth in SEQ ID NO: 30.

In some embodiments, the polynucleotide comprises the nucleic acid sequence:

(SEQ ID NO: 31)
ATGGCCCTCCCTCTTCTTGTCAACGCTGGTTTATTGGCTTTACCGATCGC
AGGTAGCATCGGC.

In some embodiments, the polynucleotide encoding a signal peptide comprises or consists of a nucleic acid sequence set forth in SEQ ID NO: 31.

In some embodiments, the polynucleotide comprises the nucleic acid sequence:

(SEQ ID NO: 32)
ATGGAGAAAGGAGGGTCAGTATTTAGTAGCAGGGTATGGGGGATTAAGCG
AGGATCTAATAGTAGGTTTTTCTTCGGG.

In some embodiments, the polynucleotide encoding a signal peptide comprises or consists of a nucleic acid sequence set forth in SEQ ID NO: 32.

In some embodiments, the polynucleotide of the invention further comprises a nucleic acid sequence encoding a signal peptide as set forth in any one of SEQ ID Nos: 24-32. In some embodiments, the signal peptide is encoded by a nucleic acid sequence comprising at least 70%, at least 80%, at least 90%, at least 95%, at least 97%, at least 99%, or 100% homology or identity to any one of SEQ ID Nos: 24-32, or any value and range therebetween. Each possibility represents a separate embodiment of the invention. In some embodiments, the polynucleotide comprises a nucleic acid sequence with 70% to 100% homology or identity to any one of SEQ ID Nos: 24-32. Each possibility represents a separate embodiment of the invention.

Methods for obtaining a codon preference of a cell, and modifying a sequence of interested so as to achieve improved expression are common and would be apparent to one of ordinary skill in the art of molecular biology and genetic engineering.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 4, at least 5, or at least 6 different nucleic acid sequences, or any value and range therebetween. Each possibility represents a separate embodiment of the invention. In some embodiments, the polynucleotide comprises 2-3, 2-4, 2-5, 2-6, 3-4, 3-5, 3-6, 4-5, 4-6, or 5-6 different nucleic acid sequences.

In some embodiments, the polynucleotide is a plurality of polynucleotide molecules, wherein each of the plurality of the polynucleotide molecules comprises a different nucleic acid sequence.

In some embodiments, the polynucleotide encodes a polypeptide characterized by having cobalamin binding activity or capability. In some embodiments, the polynucleotide encodes a polypeptide characterized by having specific binding affinity to cobalamin. In some embodiments, the polynucleotide encodes a polypeptide characterized by having a cobalamin binding domain. In some embodiments, the polynucleotide encodes a polypeptide characterized by having anti-acidic property. In some embodiments, anti-acidic property comprises protecting cobalamin from undergoing degradation or decay under acidic pH or acidic environment, such as, but not limited to in a stomach of a mammal. In some embodiments, complexation or binding of cobalamin with the polypeptide encoded by the polynucleotide as disclosed herein, reduces or inhibits the degradation or decay of cobalamin under acidic pH or acidic environment, such as, but not limited to, in a stomach of a mammal. In some embodiments, the polynucleotide encodes a transcobalamin. In some embodiments, the polynucleotide encodes a functional analog of transcobalamin. In some embodiments, the polynucleotide encodes a portion or a fragment of transcobalamin. In some embodiments, the polynucleotide encodes a domain of transcobalamin. In some embodiments, the encoded fragment, portion, domain, or analog, is capable of binding cobalamin, protecting, inhibiting, or reducing the degradation or decay of cobalamin under acidic pH or acidic environment, such as, but not limited to, in a stomach of a mammal. In some embodiments, the transcobalamin is a transcobalamin derived from a livestock animal. In some embodiments, the transcobalamin is a transcobalamin derived from a livestock selected from: cattle, sheep, goat, horse, swine, camel, or any combination thereof.

In some embodiments, enterocytes comprise the enterocytes of the small intestine. In some embodiments, the enterocytes are or comprises the enterocytes of the ileum.

According to some embodiments, there is provided an artificial nucleic acid molecule comprising the polynucleotide disclosed herein. According to some embodiments, there is provided a synthetic nucleic acid molecule comprising the polynucleotide disclosed herein.

In some embodiments, the artificial vector comprises a plasmid. In some embodiments, the artificial vector is an expression vector. In some embodiments, the artificial vector is for use in expressing a transcobalamin encoding nucleic acid sequence as disclosed herein. In some embodiments, the artificial vector is for use in heterologous expression of a transcobalamin encoding nucleic acid sequence as disclosed herein in a cell, or an organism. In some embodiments, the artificial vector is for use in producing or the production of a transcobalamin polypeptide, transcobalamin polypeptide-cobalamin complex, or both, as disclosed herein, in a cell, or an organism.

In some embodiments, an organism is or comprises a unicellular organism.

As used herein, the terms “artificial” and “synthetic” are used herein interchangeably.

Expressing of a polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II.

Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hspl7.5-E or hspl7.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. Sec, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a nucleic acid sequence encoding the transcobalamin polypeptide as described herein.

According to some embodiments, there is provided a transcobalamin polypeptide encoded by: (a) the polynucleotide disclosed herein; or (b) the artificial vector disclosed herein; or the plasmid disclosed herein.

In some embodiments, the transcobalamin polypeptide is an isolated transcobalamin polypeptide.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, a peptide comprises a chain of 2 to 50 amino acids. In some embodiments, a peptide is up to 50 amino acids long. In some embodiments, a “polypeptide” and/or a “protein” comprises at least 50 amino acids. In some embodiments, a “polypeptide” and/or a “protein” comprises multiple peptide subunits.

As used herein, the terms “isolated polypeptide” refers to a protein that is essentially free from contaminating cellular components, such as carbohydrate, lipid, or other proteinaceous impurities associated with the nucleic acid in nature. Typically, a preparation of an isolated polypeptide contains the protein in a highly purified form, e.g., at least about 80% pure, at least about 90% pure, at least about 95% pure, greater than 95% pure, or greater than 99% pure. In some embodiments, the isolated polypeptide is a synthesized polypeptide. Synthesis of a polypeptide is well known in the art and may be performed, for example, by heterologous expression in a transformed cell, such as exemplified herein.

In some embodiments, the transcobalamin polypeptide comprises or consists of the amino acid sequence:

(SEQ ID NO: 3)
MRPSGQLPLTGLLFFSLIPSQLCQICVVNETNFWRLEPLINTMLNANYTK
GTLGLNVLLTLRLVGIKNQSLEQQLSQQIKEDINSGEMELTSGELALGIL
GLGACENQDEEFIRGARLVSKLEAKFQAEIQNMEAHDGNPLTNYYQLSLA
LLALCLFNGSYSVTSVTCYFTPENKNYYFGDQFSVDTGAMAVLALTCVQK
NTQKNMDMNAEGKISNYVASLANKIQAERKQGLFGNIFSTGEAMQALFVS
SDYYKNEANCRETLRAVFDNISRGAFYLPITAAQILPALMGKTYLDVTNP
SCGLNPVKENTSTEKPGTVTPTTAPLNILVKYSVRINKTSHTEVTVRKGS
VFLDVMKAAQEKNETLFRFTVEETSWGPYITSVQGIKASNNDRTYWKLLS
NGQPLSQGAGSHVVQNGDNLEVRWSKY.

In some embodiments, the transcobalamin polypeptide comprises an amino acid sequence having at least 70%, at least 75%, at least 80%, at least 85%, at least 90%, at least 95%, at least 97%, at least 99%, or 100%, homology to the amino acid sequence set forth in SEQ ID NO: 3, or any value and range therebetween. Each possibility represents a separate embodiment of the invention. In some embodiments, the transcobalamin polypeptide comprises an amino acid sequence having 70-100% homology to the amino acid sequence set forth in SEQ ID NO: 3.

In some embodiments, a transcobalamin 1 polypeptide comprises or consists of an amino acid sequence set forth in SEQ ID NO: 3.

In some embodiments, the transcobalamin polypeptide comprises or consists of the amino acid sequence:

(SEQ ID NO: 4)
MGHLGALLFLLGGLGALANICEITEVDSTLVERLGQRLLPWMDRLSQEQL
NPSIYVGLRLSSLQAGAKEAHYLHSLKLSYQQSLLRPASNKDDNDSEAKP
SMGQLALYLLALRANCEFIGGRKGDRLVSQLKRFLEDEKRAIGHNHQGHP
RTSYYQYSLGILALCVHQKRVHDSVVGKLLYAVEHKPHLLQDHVSVDTMA
MAGMAFSCLELSNLNPKQRNRINLALKRVQEKILKAQTPEGYFGNVYSTP
LALQLLMGSLRPSVELGTACLKAKAALQASLQHKTFQNPLMISQLLPVLN
QKSYVDLISPDCQAPRALLEPALETPPQAKVPKFIDVLLKVSGISPSYRH
SVSVPAGSSLEDILKNAQEHGRFRFRTQASLSGPFLTSVLGRKAGEREFW
QVLRDPDTPLQQGIADYRPKDGETIELRLVGW.

In some embodiments, the transcobalamin polypeptide comprises an amino acid sequence having at least 70%, at least 75%, at least 80%, at least 85%, at least 90%, at least 95%, at least 97%, at least 99%, or 100%, homology to the amino acid sequence set forth in SEQ ID NO: 4, or any value and range therebetween. Each possibility represents a separate embodiment of the invention. In some embodiments, the transcobalamin polypeptide comprises an amino acid sequence having 70-100% homology to the amino acid sequence set forth in SEQ ID NO: 4.

In some embodiments, a transcobalamin 2 polypeptide comprises or consists of an amino acid sequence set forth in SEQ ID NO: 4.

In some embodiments, the transcobalamin polypeptide comprises or consists of the amino acid sequence:

(SEQ ID NO: 5)
QICVVNETNFWRLEPLINTMLNANYTKGTLGLNVLLTLRLVGIKNQSLEQ
QLSQQIKEDINSGEMELTSGELALGILGLGACENQDEEFIRGARLVSKLE
AKFQAEIQNMEAHDGNPLTNYYQLSLALLALCLFNGSYSVTSVTCYFTPE
NKNYYFGDQFSVDTGAMAVLALTCVQKNTQKNMDMNAEGKISNYVASLAN
KIQAERKQGLFGNIFSTGEAMQALFVSSDYYKNEANCRETLRAVFDNISR
GAFYLPITAAQILPALMGKTYLDVTNPSCGLNPVKFNTSTEKPGTVTPTT
APLNILVKYSVRINKTSHTEVTVRKGSVFLDVMKAAQEKNETLFRFTVEE
TSWGPYITSVQGIKASNNDRTYWKLLSNGQPLSQGAGSHVVQNGDNLEVR
WSKY

In some embodiments, the transcobalamin polypeptide comprises an amino acid sequence having at least 70%, at least 75%, at least 80%, at least 85%, at least 90%, at least 95%, at least 97%, at least 99%, or 100%, homology to the amino acid sequence set forth in SEQ ID NO: 5, or any value and range therebetween. Each possibility represents a separate embodiment of the invention. In some embodiments, the transcobalamin polypeptide comprises an amino acid sequence having 70-100% homology to the amino acid sequence set forth in SEQ ID NO: 5.

In some embodiments, a transcobalamin 1 polypeptide comprises or consists of an amino acid sequence set forth in SEQ ID NO: 5.

In some embodiments, the transcobalamin polypeptide comprises or consists of the amino acid sequence:

(SEQ ID NO: 6)
NICEITEVDSTLVERLGQRLLPWMDRLSQEQLNPSIYVGLRLSSLQAGAK
EAHYLHSLKLSYQQSLLRPASNKDDNDSEAKPSMGQLALYLLALRANCEF
IGGRKGDRLVSQLKRFLEDEKRAIGHNHQGHPRTSYYQYSLGILALCVHQ
RVHDSVVGKLLYAVEHKPHLLQDHVSVDTMAMAGMAFSCLELSNLNPKQR
NRINLALKRVQEKILKAQTPEGYFGNVYSTPLALQLLMGSLRPSVELGTA
CLKAKAALQASLQHKTFQNPLMISQLLPVLNQKSYVDLISPDCQAPRALL
EPALETPPQAKVPKFIDVLLKVSGISPSYRHSVSVPAGSSLEDILKNAQE
HGRFRFRTQASLSGPFLTSVLGRKAGEREFWQVLRDPDTPLQQGIADYRP
KDGETIELRLVGW.

In some embodiments, the transcobalamin polypeptide comprises an amino acid sequence having at least 70%, at least 75%, at least 80%, at least 85%, at least 90%, at least 95%, at least 97%, at least 99%, or 100%, homology to the amino acid sequence set forth in SEQ ID NO: 6, or any value and range therebetween. Each possibility represents a separate embodiment of the invention. In some embodiments, the transcobalamin polypeptide comprises an amino acid sequence having 70-100% homology to the amino acid sequence set forth in SEQ ID NO: 6.

In some embodiments, a transcobalamin 2 polypeptide comprises or consists of an amino acid sequence set forth in SEQ ID NO: 6.

In some embodiments, the transcobalamin polypeptide further comprises a signal peptide. In some embodiments, the transcobalamin polypeptide comprises a signal peptide and a cobalamin binding polypeptide.

In some embodiments, the signal peptide comprises or consists the amino acid sequence: MKFFAIAALFAAAAVAQPNVISKR (SEQ ID NO: 7).

In some embodiments, the signal peptide comprises or consists the amino acid sequence: MKFFAIAALFAAAAVA (SEQ ID NO: 8).

In some embodiments, the signal peptide comprises or consists the amino acid sequence: MMVAWWSLFLYGLQVAAPALA (SEQ ID NO: 33).

In some embodiments, the signal peptide comprises or consists the amino acid sequence: MRNNLLFSLNAIAGAVAHPSFPIHKR (SEQ ID NO: 34).

In some embodiments, the signal peptide comprises or consists the amino acid sequence: MPWAGLATLFLAVAGVHG (SEQ ID NO: 35).

In some embodiments, the signal peptide comprises or consists the amino acid sequence: MQFSVAAVLALATAVAA (SEQ ID NO: 36).

In some embodiments, the signal peptide comprises or consists the amino acid sequence: MSHVEHLRHTALQLRSPLFTCPHLPV (SEQ ID NO: 37).

In some embodiments, the signal peptide comprises or consists the amino acid sequence: MTPSLSKLVALSLFLGTALG (SEQ ID NO: 38).

In some embodiments, the signal peptide comprises or consists the amino acid sequence: MALPLLVNAGLLALPIAGSIG (SEQ ID NO: 39).

In some embodiments, the signal peptide comprises or consists the amino acid sequence: MEKGGSVFSSRVWGIKRGSNSRFFFG (SEQ ID NO: 40).

In some embodiments, the signal peptide comprises an amino acid sequence with at least 70%, at least 80%, at least 90%, at least 95%, at least 97%, at least 99%, or 100% homology or identity to an amino acid sequence selected from the group consisting of: SEQ ID NO: 7, SEQ ID NO: 8, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and any combination thereof. Each possibility represents a separate embodiment of the invention. In some embodiments, the polynucleotide comprises a nucleic acid sequence with 70% to 100% homology or identity to an amino acid sequence selected from the group consisting of: SEQ ID NO: 7, SEQ ID NO: 8, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and any combination thereof. Each possibility represents a separate embodiment of the invention.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

In some embodiments, the polypeptide comprises or is characterized by having an activity selected from: protecting cobalamin from acid degradation, transporting cobalamin from enterocytes to peripheral tissues, or any combination thereof.

According to some embodiments, there is provided a homogenate, lysate, extract, any portion thereof, any fraction thereof, or any combination thereof, derived or obtained from a recombinant cell as disclosed herein.

In some embodiments, the extract comprises the transcobalamin polypeptide, cobalamin, transcobalamin-cobalamin complex, or any combination thereof.

In some embodiments, the extract comprises the polynucleotide of the invention, a DNA molecule as disclosed herein, a protein as disclosed herein, or any combination thereof.

### Compositions

According to some embodiments, there is provided a composition comprising the recombinant cell of the invention, or any material derived or processed therefrom. In some embodiments, there is provided a composition comprising a recombinant transcobalamin, derived from the recombinant cell of the invention, and cobalamin. In some embodiments, the composition comprises a complex comprising cobalamin bound to a recombinant transcobalamin polypeptide.

In some embodiments, the composition further comprises an acceptable carrier.

In some embodiments, a material derived or processed from the recombinant cell of the invention comprises any one of: cell secretome, cell lysate, cell extract, cell homogenate, a portion thereof, a fraction thereof, or any combination thereof, of the recombinant cell.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

As used herein, the term “secretome” encompasses any compound and/or molecule, or a plurality thereof being secreted from a cell, e.g., a recombinant cell, or a transgenic/transfected/transformed cell as described herein, into the extra environment of the cell. Such secretome may include: a polypeptide (e.g., transcobalamin), a vitamin (e.g., cobalamin), a metabolite, a lipid, a carbohydrate, and extracellular vesicle, a complex thereof, or any combination thereof.

In some embodiments, the material derived or processed from the recombinant cell comprises the transcobalamin polypeptide disclosed herein, cobalamin, or both. In some embodiments, the transcobalamin polypeptide is bound to cobalamin.

According to some embodiments, there is provided a food composition comprising the herein disclosed composition. In some embodiments, the food composition is enriched with the herein disclosed composition. In some embodiments, the food composition is enriched with the transcobalamin polypeptide disclosed herein, cobalamin, or a complex comprising same (transcobalamin-cobalamin complex).

In some embodiments, the food composition further comprises a milk protein or a plurality thereof.

In some embodiments, the milk protein or a plurality thereof is obtained or derived from a fungal cell, or any material derived or processed therefrom.

In some embodiments, the food composition comprising a material derived or processed from a fungal cell comprises any one of: cell secretome, cell lysate, cell extract, cell homogenate, a portion thereof, a fraction thereof, or any combination thereof, of the fungal cell.

In some embodiments, the fungal cell comprises a Trichoderma reesei cell, an Aspergillus oryzae cell, or a combination thereof.

In some embodiments, the food composition comprises a milk protein or a plurality thereof obtained or derived from a Trichoderma reesei cell, an Aspergillus oryzae cell, or a combination thereof.

In some embodiments, the food composition is a solid food composition. In some embodiments, the food composition is a semi solid composition. In some embodiments, the food composition is a liquid food composition.

In some embodiments, the food composition is an edible composition. In some embodiments, the food composition is a nutraceutical composition.

In some embodiments, the food composition is selected from: a milk, a yogurt, a cheese, a butter, a caseinate, a cream, an infant formula, an ice cream, a frozen custard, a cottage cheese, a cream cheese, a crème fraiche, or a curd.

As used herein, the term “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8^{th} Ed., Gilman et al. Eds. Pergamon Press (1990); Remington's Pharmaceutical Sciences, 18^{th} Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Production

According to some embodiments, there is provided a method for producing a transcobalamin-cobalamin complex.

In some embodiments, the method for producing a transcobalamin-cobalamin complex comprises: (a) culturing a cell comprising an exogenous polynucleotide comprising a nucleic acid sequence encoding a mammalian transcobalamin polypeptide, (b) culturing the cell from step (a) such that the mammalian transcobalamin polypeptide encoded from the exogenous polynucleotide is expressed; and (c) contacting the mammalian transcobalamin with cobalamin, thereby producing a transcobalamin-cobalamin complex.

In some embodiments, contacting is in conditions that allow the binding of transcobalamin to cobalamin. In some embodiments, contacting is in conditions that allow the formation of the complex transcobalamin-cobalamin. In some embodiments, contacting is in about 37° C. In some embodiments, contacting is for at least 10 minutes.

In some embodiments, the method comprises: (a) culturing a cell comprising a polynucleotide comprising a nucleic acid sequence encoding a transcobalamin polypeptide, as disclosed herein, wherein the cell comprises cobalamin; and (b) culturing the cell from step (a) such that the transcobalamin polypeptide encoded from the polynucleotide is expressed, thereby producing a transcobalamin-cobalamin complex.

In some embodiments, the method comprises: (a) providing a cell comprising an artificial vector comprising the polynucleotide disclosed herein; and (b) culturing the cell from step (a) such that a transcobalamin polypeptide encoded by the artificial vector is expressed.

In some embodiments, the cell is the cell disclosed herein.

In some embodiments, the cell is a transgenic cell or transfected cell comprising the polynucleotide as disclosed herein, or a plurality thereof, as disclosed herein.

In some embodiments, the cell is a transgenic cell or a transfected cell comprising an artificial nucleic acid molecule or vector comprising the polynucleotide, as disclosed herein.

In some embodiments, the cell is a transgenic cell, or a cell transfected with a DNA molecule or polynucleotide as disclosed herein.

In some embodiments, the method further comprises a step preceding step (a) comprising introducing or transfecting the cell with the polynucleotide disclosed herein.

In some embodiments, the method further comprises a step (c) comprising: (i) separating the cultured cell from a medium wherein the cell is cultured, and extracting the separated medium; (ii) extracting the cell; or (iii) both (i) and (ii), thereby, obtaining an extract of the separated medium, an extract of the cell, or both.

According to some embodiments, there is provided a method for preparing a food composition.

In some embodiments, the method comprises mixing the extract disclosed herein with a milk protein or a plurality thereof, as disclosed herein.

According to some embodiments, there is provided an extract of: (a) a separated medium; (b) a cell; (c) or both (a) and (b), obtained according to the herein disclosed method.

In some embodiments, the extract obtained according to the herein disclosed method comprises a transcobalamin-cobalamin complex.

According to some embodiments, there is provided a composition comprising the herein disclosed extract, and a nutraceutically acceptable carrier.

According to some embodiments, there is provided a method for obtaining an extract from a cell.

In some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide/DNA molecule disclosed herein into a cell; or modifying the genome of a cell to include the polynucleotide/DNA molecule disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

Method for separating cell from a medium are common and may include, but not limited to, centrifugation, ultracentrifugation, or other, as would be apparent to one of ordinary skill in the art.

According to some embodiments, there is provided a medium or a portion thereof separated from a cultured cell or, obtained according to the herein disclosed method.

In some embodiments, a portion comprises a fraction or a plurality thereof.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (cds), “Strategies for Protein Purification and Characterization—A Laboratory Course Manual” CSHL Press (1996); all of which are incorporated by reference. Other general references are provided throughout this document.

### Materials and Methods

The coding sequences of bovine TC1 and TC2 were optimized for expression in A. Oryzae. The optimized sequences were integrated into the synthetic expression system to generate expression cassettes. These expression cassettes, flanked by ˜1 kb sequences located upstream and downstream of the A. Oryzae amylase C (amyC) gene (designated amyC 5′-flank and amyC 3′-flank, respectively), were each cloned into a backbone plasmid. The resulting plasmids were propagated in E. coli dh5a cells, extracted, digested by restriction enzymes, and the expression cassettes were agarose gel purified. The linearized DNA cassettes were transformed into A. Oryzae using a previously described method. Positive transformant colonies were picked and PCR-validated, then transferred to an additional round of isolation by single-spore plating and PCR validation. Mycelia from isolated clones were grown in 50 ml of BMDY medium with 2% glucose, at 280 C and shaking at 150 rpm for 3 days. The extracellular medium was sampled for SDS-PAGE gel (14% tris-glycine gel, 200V 25 min). For testing of TC2 expression, the size-separated proteins were transferred into Nitro-Cellulose membrane using a Trans-Blot Turbo Transfer System. The membranes were incubated Rabbit anti TC2 antibody (1:1000) and anti-rabbit secondary antibody (1:10000), to specifically detect TC2. In addition, the blotted membranes were analyzed by mass spectrometric proteomic analysis.

### Example 1

The invention is at least partially, focused on producing a milk protein in food-safe microbial expression microflora organism, with a high level of heterologous protein production, such as the filamentous fungi Trichoderma, Aspergillus and yeast.

An integrated AI/machine learning precision fermentation approach was tailored to produce transcobalamin. Advanced algorithms modelling gene expression (e.g., transcription, translation, co-translational folding and mRNA stability) were developed to create ML/AI system. The models combined approaches from biophysics, information theory, machine learning, and computational molecular evolution. The system was fed with large scale experimental data based on next generation sequencing (NGS) and Ribo Seq. Various synthetic biology approaches were used for screening variants and for decoupling their expression within and or/secretion from an endogenous system.

To build the ideal transcobalamin genes, more than 100 genomes were analyzed, and then the optimized genes were introduced into the host's DNA. A synthetic system, created and tuned to regulate the transcobalamin expression independently of the host's metabolic status, was used to selectively secrete transcobalamin, while blocking the secretion of undesirable background proteins. The medium was then harvested, and transcobalamin protein was purified with more than 98% purity.

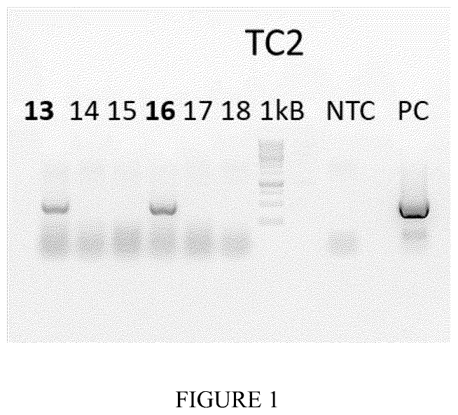

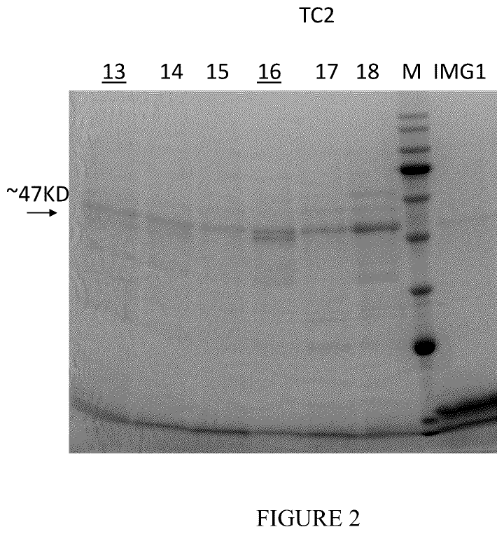

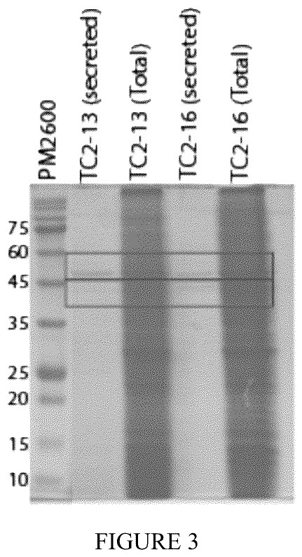

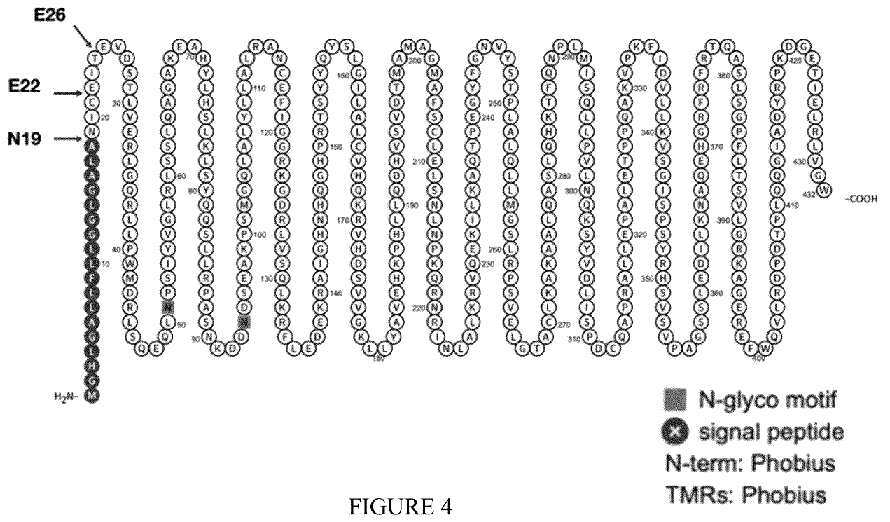

Open reading frame (ORF) polymerase chain reaction (PCR) was performed to verify genomic DNA of TC2 (FIG. 1). Western blot analysis confirmed expression of TC2 (FIG. 2). Proteomics analysis confirmed the expression of TC2 in A. orizae. The samples were separated by 12% SDS-PAGE and stained with Coomassie (FIG. 3). The marked regions were excised from each lane and were subjected to in-gel digestion with trypsin. The resulting peptides were desalted and submitted to LC-MS/MS. The RAW MS/MS data was searched against sequence database composed of all of Aspergillus oryzae sequences downloaded from Uniprot supplemented with several relevant bovine sequences and known contaminating proteins (cRAP) using FragPipe proteomics analysis pipeline. The main conclusions from this analysis were that TC2 was expressed and secreted from A. orizac. In addition to TC2, A. orizae mainly secretes alpha-amylase A type-1/2 (Uniprot ID: POC1B3) and maybe also enolase A (Uniprot ID: Q12560). It was also found that while TC2 was identified in its secreted form, without the signal peptide, the sequence expressed also consisted of 2 additional N-terminal cleavage sites that lead to the appearance of a protein that starts at position 22 or at position 25, as demonstrated in FIG. 4.

The protein was subsequently examined in biochemical tests to show effective B12 binding. The entire process was scalable to produce the protein in large batches. Afterwards, the inventors feedbacked the expression information (RNAseq) to the machine learning to create better constructs until reaching optimal transcobalamin expression.

### Example 2

Absorption tests are conducted ex vivo, showing that absorption of B12 is much more efficient with recombinant TC loaded with B12, compared to free B12.

Animal studies are conducted so as to determine and demonstrate efficient absorption with B12-TC complex.

MAC-T, HeLa, and C127 cells, free cyanocobalamin (vitamin B12) (Sigma) or B12 in the form of a trans-cobalamin-B12 complex are added to the culture medium at concentrations ranging from 1 to 9 ng of B12/ml. Cells are cultured in the presence of B12 for 16 h, then washed three times with phosphate buffered saline (PBS). The cells are then removed by trypsin treatment, centrifuged at 220 g and washed twice with PBS. The cells are resuspended in 0.3 ml PBS and chilled on ice for 30 min prior to lysis by sonication on ice using a probe in three 10 sec bursts. Cell lysates are centrifuged at 800 g for 20 min and the protein concentration and vitamin B12 content of the supernatant are determined using a Bradford assay kit (Bio-Rad, Hercules, CA).

For differentiated Caco-2 cells, B12 are added in concentrations ranging from 0 to 40 ng/ml. The B12 are added either to the apical side of the polarized epithelial cell layer (on top of the inserts) or the basolateral side (in the bottom of the well). The vitamin B12 is added either in the form of free B12, intrinsic factor (IF)-B12 complex, or transcobalmin-B12 complex. The IF-B12 complex is prepared by pre-incubating a 1:1 molar ratio of human recombinant IF and B12 (Cobento Biotech, Aarhus, Denmark) for 1 h, at room temperature. The transcobalamin-B12 complex in the pre-existing complex isolated recombinant TC and HC. The Caco-2 cells are incubated and processed as described above.

TC: B12 molar ratio, affinity, and conditions of release, are determined.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A composition comprising cobalamin and a recombinant cell comprising an exogenous polynucleotide comprising a nucleic acid sequence encoding a mammalian transcobalamin (TC) polypeptide or a material derived or processed from said recombinant cell.

2. The composition of claim 1, wherein said recombinant cell is any one of: a unicellular organism, a cell of a multicellular organism, and a cell in a culture, optionally wherein said unicellular organism is a bacterium or a fungus and optionally wherein said fungus is selected from the group consisting of: Aspergillus oryzae and Trichoderma reesei.

3. (canceled)

4. (canceled)

5. The composition of claim 1, wherein said recombinant cell is Aspergillus oryzae.

6. The composition of claim 1, wherein said TC polypeptide comprises: transcobalamin 1 (haptocorrin), transcobalamin 2, or both.

7. The composition of claim 1, wherein said material derived or processed from said recombinant cell comprises any one of: cell secretome, cell lysate, cell extract, cell homogenate, a portion thereof, a fraction thereof, and any combination thereof, of said recombinant cell.

8. The composition of claim 1, wherein said mammalian TC comprises an amino acid sequence having at least 70% homology to an amino acid sequence set forth in any one of SEQ ID Nos: 3-6.

9. The composition of claim 1, comprising said TC polypeptide bound to said cobalamin.

10. The composition of claim 1, wherein said exogenous polynucleotide encoding said mammalian TC polypeptide is codon optimized for expression in said recombinant cell.

11. The composition of claim 1, wherein said mammalian TC further comprises a signal peptide comprising an amino acid sequence having at least 70% homology to an amino acid sequence set forth in any one of SEQ ID Nos: 7, 8, and 33-40.

12. The composition of claim 1, wherein said exogenous polynucleotide comprises a nucleic acid sequence having at least 70% identity to a nucleic acid sequence set forth in any one of SEQ ID Nos: 1, 2, and 9-22.

13. The composition of claim 1, wherein said exogenous polynucleotide further comprises a nucleic acid sequence encoding a signal peptide, wherein said exogenous polynucleotide comprises a nucleic acid sequence having at least 70% identity to a nucleic acid sequence set forth in any one of SEQ ID Nos: 24-32.

14. The composition of claim 1, further comprising a nutraceutically acceptable carrier.

15. The composition of claim 1, being a food composition.

16. The food composition of claim 15, further comprising a milk protein or a plurality thereof, being derived from a fungal cell, a bacterial cell, or any combination thereof, and optionally wherein said food composition being selected from the group consisting of: a milk, a yogurt, a cheese, a butter, a caseinate, a cream, an infant formula, an ice cream, a frozen custard, a cottage cheese, a cream cheese, a crème fraiche, and a curd.

17. (canceled)

18. A method for producing a composition comprising a TC-cobalamin complex, the method comprising:
a) culturing a recombinant cell comprising an exogenous polynucleotide comprising a nucleic acid sequence encoding a mammalian TC polypeptide;
b) culturing said cell from step (a) such that the mammalian TC polypeptide encoded from said exogenous polynucleotide is expressed; and;
c) contacting said mammalian TC with cobalamin,
thereby producing the composition comprising a TC-cobalamin complex.

19. The method of claim 18, wherein said recombinant cell further comprises said cobalamin, and optionally wherein said recombinant cell is a fungal cell or a bacterial cell.

20. (canceled)

21. The method of claim 18, further comprising a step preceding step (a) comprising introducing or transfecting a cell with said exogenous polynucleotide, thereby obtaining said recombinant cell.

22. The method of claim 18, wherein any one of: (i) said TC polypeptide comprises: transcobalamin 1 (haptocorrin), transcobalamin 2, or both; (ii) said mammalian TC comprises an amino acid sequence having at least 70% homology to an amino acid sequence set forth in any one of SEQ ID Nos: 3-6; (iii) said composition is a dairy composition; and (iv) any combination of (i) to (iii).

23. (canceled)

24. The method of claim 18, further comprising a step after step (b) and before step (c), comprising:
a) separating said cultured cell from a medium wherein said cell is cultured; and
b) extracting any one of:
i. said separated medium
ii. said separated cell; and
iii. both (i) and (ii),
and optionally wherein said method further comprises a step preceding step (c) comprising isolating said TC polypeptide from said extract.

25. (canceled)

26. The method claim 18, further comprising a step (d) comprising mixing said TC-cobalamin complex with a milk protein or plurality thereof.

27. (canceled)
